(19) United States
(12) Patent Application Publication
(10) Pub. No.: US 2026/0034046 A1
(43) Pub. Date: Feb. 5, 2026

(54) ANHYDROUS HAIR TREATMENT COMPOSITION COMPRISING CATIONIC SURFACTANT AND FATTY ALCOHOL

(71) Applicant: L'OREAL, Paris (FR)

(72) Inventors: XianZhi ZHOU, Millburn, NJ (US); Jun LIANG, Brooklyn, NY (US)

(73) Assignee: L'OREAL, Paris (FR)

(21) Appl. No.: 18/789,328

(22) Filed: Jul. 30, 2024

(51) Int. Cl. A61K 8/92; A61K 8/34; A61K 8/37; A61K 8/42; A61Q 5/12

(52) U.S. Cl. CPC A61K 8/922; A61K 8/342; A61K 8/375; A61K 8/42; A61Q 5/12; A61K 2800/31

(57) ABSTRACT

The instant disclosure is drawn to an anhydrous hair treatment composition comprising: (a) one or more vegetable oils, wherein one of the one or more vegetable oils is castor oil; (b) one or more cationic surfactants; (c) one or more fatty alcohols; and (d) optionally, one or more sensorially active agents. In addition, the composition is essentially free from mono-alcohols and polyhydric alcohols having from 1 to 6 carbon atoms and essentially free from esters of alpha hydroxy acids and acetal and ketals having 10 carbon atoms or less. The anhydrous hair treatment composition is particularly useful in methods for treating hair.

### FIELD OF THE DISCLOSURE

The present disclosure relates to anhydrous hair treatment compositions comprising cationic surfactants and fatty alcohols, and to methods for treating hair with the compositions. The compositions and methods are useful for smoothing the hair, treating split-ends, and controlling frizz.

### BACKGROUND

Many consumers desire to use cosmetic and care compositions that enhance the appearance of hair, e.g., by changing the color, style, and/or shape of the hair, and/or by imparting various cosmetic properties to hair, such as shine and conditioning. Hair can become dry or damaged for various reasons, e.g., weather exposure, poor nutrition, mechanical treatments (e.g. brushing hair), styling treatments using chemicals, dying, heat, nutrition, etc. Even cleansing products tend to remove hair's natural oils causing dryness, which can lead to a dull appearance and to split ends. Individuals with curly hair can also encounter additional challenges when caring for their hair. Curly hair more easily tangles and can be more difficult to manage.

Environmental factors, such as salt water, sunlight, and heat, and chemical treatments are known to damage hair. Various types of chemical treatments include, for example, hair bleaching and coloring, permanents, waving products, and relaxing treatments (straightening treatments). These chemical treatments change the look of hair by changing its physical structure, which inevitably causes a certain degree of damage to the hair. Damaged hair is characterized by unnatural changes to the protein structure of the individual hair strands or shafts. Damage results in split ends, dry straw-like hair, hair that is easily broken, and hair that is “frizzy” and unmanageable. Because the visible portion of hair is dead, it has no ability to regenerate itself.

There are numerous over the counter and salon treatments that purport to repair damaged hair. Silicones, for example, have been used to add lubricity and shine to hair. Silicones work by coating the cuticle, the outermost layer of the hair fiber. Silicones are often found in conditioners because they increase slip, help lock-in moisture, and leave the hair feeling soft. The increased “slip” helps ease detangling of the hair. Silicones are also used for their anti-frizz properties because they can form a coating on the hair that resists humidity that causes frizz. However, silicones can build-up on the hair leaving a residue that is difficult to wash out. The residue ultimately prevents moisture from absorbing into the hair, thereby actually causing dryness and damage over time. In addition, silicones can weigh down the hair, especially fine hair, causing it to lose volume and bounce.

Products that condition hair include rinse-out conditioners, leave-in conditioners, deep conditioners, and co-washes. Rinse-out conditioners are the most common type of conditioning product. They are applied after cleansing the hair with a shampoo and are rinsed from the hair before drying and styling the hair. Rinse-out conditioners typically remain in the hair for only a few minutes before being rinsed from the hair yet they are suitable for most hair types and can be used daily or as needed.

Leave-in conditioners are usually applied to the hair after washing the hair with a shampoo and are not rinsed out. They remain on the hair during drying and styling and are designed to provide long-lasting moisturization and nourishment, making them a popular choice for dry or damaged hair. Leave-in conditioners can also offer additional benefits such as heat protection, frizz control, and detangling. They are especially useful for those who style their hair frequently or need extra hydration throughout the day.

All-in-one shampoo/conditioner products, also known as “co-wash” products or “conditioning shampoos,” attempt to combine the cleansing benefits of a shampoo with the conditioning properties of a conditioner. They proport to cleanse the hair while simultaneously conditioning the hair, making them a convenient and simplified hair care routine. Cleansing compositions, like shampoos, are designed to remove sebum, oil, and debris from the hair, which is antithetical to conditioning. Many conditioning products are designed to impart oil and other conditioning agents to the hair. Furthermore, effective shampoos cause the cuticle of the hair to open helping to release and remove debris. Effective conditioning products, on the other hand, are designed to close the cuticle to prevent release of moisture and to provide added protection to the hair. A single product cannot open and close the cuticle at the same time.

Conditioning products on the market appear in various forms, for example, solutions, lotions, creams, sprays, oils, and even aerosols. Regardless of the form, consumers seek products that are lightweight so they do not weigh down the hair or leave the hair with a greasy or oily texture. Conditioned hair should look natural and healthy, have a pleasant shine, and flow naturally. Ideal conditioning products moisturize the hair, provide protection from heat and other environmental damage, and control frizz and flyaways.

The formulation of environmentally-friendly cosmetic products, which are designed and developed considering environmental issues, is also becoming a major goal in an effort to meet global challenges. It is therefore essential to propose more sustainable compositions, preparation processes and ingredients to address these environmental concerns. In this context, it is important to develop new cosmetic compositions with a better carbon footprint, particularly by promoting the use of renewable raw materials and/or materials with a good index of naturalness and/or materials of natural origin and, more particularly, materials of plant origin while reducing the use of compounds of petrochemical origin.

### SUMMARY OF THE DISCLOSURE

The present disclosure is drawn to an anhydrous hair treatment composition that delivers instant softness, moisture, and other conditioning benefits to the hair. The composition includes castor oil, one or more cationic surfactants, and one or more fatty alcohols. The fatty alcohols behave like nonionic surfactants due to their amphipathic nature. Notably, despite containing both cationic surfactants and fatty alcohols, the composition is not an emulsion; instead, it is a homogeneous, transparent solution wherein the cationic surfactants and fatty alcohols are solubilized.

Surfactants have a polar head and a fatty tail. In emulsions, water and other polar organic compounds form an aqueous phase that interacts with the polar heads of surfactants. Fatty compounds such as oils create a hydrophobic phase of the emulsion that interacts with the fatty tails of surfactants. In the absence of water, mono-alcohols having from 1 to 6 carbon atoms, glycols having from 1 to 6 carbon atoms, and/or other polyols having from 1 to 6 carbon atoms are typically used to incorporate surfactants into oils. The inventors surprisingly discovered a unique formulation that effectively solubilizes both cationic surfactants and fatty alcohols, resulting in a transparent solution that moisturizes, softens hair, treats split-ends, and controls frizz during use with water.

The anhydrous hair treatment composition typically includes:
(a) one or more vegetable oils, wherein one of the one or more vegetable oils is castor oil in an amount of at least 10 wt. %, based on the total weight of the anhydrous hair treatment composition;
(b) one or more cationic surfactants;
(c) one or more fatty alcohols; and
(d) optionally, one or more sensorially active agents;
   all weight percentages are based on a total weight of the composition.

Upon contact with water, the anhydrous hair treatment composition undergoes a transformation into a structured lamellar arrangement. Cationic surfactants and fatty alcohols gather at the interface between the oil and water, positioning their hydrophobic tails toward the oil and their hydrophilic heads toward water molecules. This molecular alignment forms bilayers or lamellae, effectively trapping and transporting oil droplets within these layers onto the hair during application. When applied to wet or damp hair, the presence of water triggers the development of the lamellar structure. Consequently, the oil droplets become deposited onto the hair surface. Unlike conventional conditioners that utilize oils, cationic surfactants, and fatty alcohols for conditioning, the unique lamellar mechanism formed with the anhydrous hair treatment composition during use with water ensures a more efficient and thorough deposition process. Thus, this composition effectively deposits substantial amounts of oil, cationic surfactants, and other active ingredients onto the hair. Unlike other water-based conditioning products, hair treated with this anhydrous hair treatment composition feels moisturized without becoming sticky, greasy, or heavy. It imparts a lightweight, soft feel with a natural shine and a healthy appearance.

Preferably, the anhydrous hair treatment composition is free or essentially free from mono-alcohols having from 1 to 6 carbon atoms and polyhydric alcohols having from 1 to 6 carbon atoms. Furthermore, the anhydrous hair treatment composition is also, preferably, free or essentially free from esters of alpha hydroxy acids. The anhydrous hair treatment composition may additionally or alternatively be free or essentially free from acetal and ketals having 10 carbon atoms or less. A nonlimiting example of a ketal having 10 carbon atoms or less is isopropylidene glycerol. In a preferred embodiment, the anhydrous hair treatment composition is free or essentially free from isopropylidene glycerol.

The anhydrous hair treatment composition includes castor oil, typically in an amount of about 10 wt. % or more. In addition to the castor oil, the composition optionally includes one or more additional vegetable oils. In various embodiments, it is preferable to include one or more vegetable oils in addition to the castor oil. Nonlimiting examples of additional vegetable oils include arachis oil, amaranth seed oil, apricot oil, argan oil, avocado oil, babassu oil, cottonseed oil, borage seed oil, camelina oil, camellia oil, safflower oil, peanut oil, pomegranate seed oil, grapefruit seed oil, hemp oil, hazelnut oil, elderberry seed oil, currant seed oil, jojoba oil, linseed oil, macadamia nut oil, corn oil, almond oil, manila oil, evening primrose oil, olive oil, palm oil, palm kernel oil, brazil nut oil, pecan nut oil, peach kernel oil, rapeseed oil, rosehip oil, sea buckthorn fruit oil, sea buckthorn kernel oil, sesame oil, soybean oil, sunflower oil, grapeseed oil, walnut oil, wild rose oil, and/or wheat germ oil. Additional vegetable oils that are particularly useful include coconut oil, soybean oil, avocado oil, arachis oil, apricot oil, argan oil, avocado oil, babassu oil, cottonseed oil, camelina oil, camellia oil, safflower oil, and peanut oil. Preferred additional oils include coconut oil and/or soybean oil. Coconut oil is particularly preferred.

Cationic surfactants include, but are not limited to, quaternary ammonium surfactants and amidoamine surfactants. Nonlimiting examples of quaternary ammonium surfactants include cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, and tricetylmonium chloride. Nonlimiting examples of amidoamines include oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethylamine, behenamidoethyldimethylamine, arachidamidopropyldimethylamine, arachidamido-propyidiethylamine, arachidamidoethyidiethylamine, and arachidamidoethyidimethylamine.

Nonlimiting examples of fatty alcohols include branched or linear, and saturated or unsaturated fatty alcohols, which may be liquid fatty alcohols or solid fatty alcohols. Nonlimiting examples of solid fatty alcohols include stearyl alcohol, cetyl alcohol, cetearyl alcohol alcohol, myristyl alcohol, behenyl alcohol, or arachidyl alcohol. Nonlimiting examples of liquid fatty alcohols include isostearyl alcohol, oleyl alcohol, octyldodecanol, linoleyl alcohol, linolenyl alcohol, lauryl alcohol, 2-hexyldecanol, 2-heptyldecanol, enanthic alcohol (1-heptanol), caprylic alcohol (1-octanol), pelargonic alcohol (1-nonanol), or capric alcohol (1-decanol). In a preferred embodiment, the anhydrous hair treatment composition includes one or more solid fatty alcohols and one or more liquid fatty alcohols.

The anhydrous hair treatment composition optionally includes one or more polymeric oil thickening agents and/or one or more cationic conditioning polymers. Nonlimiting examples of polymeric oil thickening agents include VP/hexadecene copolymer, trimethylpentanediol/adipic acid/glycerin crosspolymer, ethylene/propylene/styrene copolymer, butylene/ethylene/styrene copolymer, VP/eicosene copolymer, bis-dioctadecylamide dimer dilinoleic acid/ethylenediamine copolymer, cetearyl dimethicone crosspolymer, hydrogenated poly(C6-20 olefin), HDI/trimethylol hexyllactone crosspolymer, dimethicone/vinyl dimethicone crosspolymer, or vinyl dimethicone/methicone silsesquioxane crosspolymer. In various embodiments, preferred polymeric oil thickening agents include hydrogenated poly(C6-20 olefin), and HDI/trimethylol hexyllactone crosspolymer.

The anhydrous hair treatment composition optionally includes one or more sensorially active agents. Nonlimiting examples of sensorially active agents include essential oils, cooling agents, terpenes, and terpenoids. Nonlimiting examples of essential oils include lavender oil, geranium oil, juniper berry oil, bay leaf oil, lemon oil, mandarin orange oil, orange oil, absinthe oil, African bluegrass oil, ajowan oil, anethi oil, angelica root oil, anise star oil, arborvitae wild oil, armoise mugwort oil, asafetida oil, Australian balm oil, basil oil, benzoin oil, bergamot calabrian oil, betel leaf oil, birch sweet oil, birch tar oil, black currant-seed oil, blood orange oil, buchu oil, cabreuva oil, cade oil, cajeput oil, calamus root oil, camphor oil, cananga oil, caraway oil, cardamom oil, carrotseed oil, cassia oil, cedarleaf oil cedarwood atles oil, cedarwood Chinese oil, cedarwood Himalayan oil, cedarwood texas oil, cedarwood Virginian oil, celery seed oil, chamomile German oil, chamomile moroc oil, chamomile roman oil, chaulmogra oil, chenopodium oil, chilly seed oil, cinnamon bark oil, cinnamon leaf oil, citral oil, citronelia Ceylon oil, clarysage oil, clementine oil, clove bud oil, clove leaf oil, cognac oil, copaiba balsam oil, coriander oil, costus root oil, coffee oil, cumin oil, curcuma oil, curry leaf oil, cypress Australian blue oil, cypress French cypriol oil, davana oil, dill seed oil, dill weed oil, elemi oil, emu oil, eucalyptus oil, fennel bitter oil, fenugreek oil, fir balsam oil, fir needle oil, flax seed organic oil, frankincense oil garlic oil, galbanum oil, ginger oil, ginger grass oil, gaia oil, grapefruit pink oil, grapefruit white oil, guaiacwood oil, helichrysum oil, horseradish oil, hydacheim oil, hyssop oil, jamarosa root oil, Himalayan wild oil, karanj seed oil, kanuka oil, katrafay oil, key lime oil, kunzea oil, kukuinut oil, lantana oil, laurel leaf oil, lemongrass oil, lemon eucalyptus oil, lemon myrtle oil, lemon 5 fold oil, lican oil, lime oil, Litsea cubeba oil, lovage leaf oil, mace oil, mandarin oil, manuka oil, marjoram oil, Melissa leaf oil, menthol liquid oil, mullein oil, myrrh oil, myrtle oil, neem oil, neroli oil, nerolina Australian oil, niaouli oil, noni oil, nutmeg oil, oat oil, onion oil, opoponax oil, orange bitter oil, orange sweet oil, origanum oil, orange 5 fold oil, palmarosa oil, parsley oil, patchouli oil, pennyroyal oil, pepper oil, peppermint oil, peru balsam oil, petitgrain oil, pimento leaf oil, pine long leaf oil, pine scotch oil, pine commercial oil, pomegranate oil, pomouwood oil, ravensara wild oil, rosalina Australian oil, rose geranium oil, rosemary oil, rosewood oil, rue oil, sage Dalmatian oil, sandalwood oil, savine oil, savory winter oil, sea buckthorn oil, spearmint oil, spikenard oil, spruce oil, sugandh kokila oil, sumac oil, tagetes oil, tangerine oil, tansy blue oil, tarragon oil, tangerine 5 fold oil, tea tree oil, tea tree lemon oil, thyme oil, tolu balsam oil, turmeric oil, valerian root oil, vanilla 10 fold oil, vetiver oil, wintergreen oil, wild verbena oil, wormwood oil, yarrow oil, and ylang ylang oil.

Nonlimiting examples of cooling agents include menthol, menthone glycerol acetal, menthyl lactate, menthyl-3-carboxylic acid N-ethylamide, 2-isopropyl-N-2,3-trimethylbutanamide, 3-menthoxypropane-1,2-diol, 2-hydroxtethyl menthyl carbonate, 2-hydroxypropyl menthyl carbonate, and isopulegol. Menthol and/or menthol derivatives are particularly useful.

Nonlimiting examples of terpenes and terpenoids include menthol, myrcene, limonene, linalool, humulene, terpinolene, camphene, terpineol, beta-caryophyllene, 1,8-cineol, anisole, anethole, eugenol, thymol, borneol, citronellol, isomenthol, camphor, camphene, carvone, geraniol, menthone, pulegone, α-pinene, β-pinene, sabinene, α-terpinene, β-bisabolene, trans-α-bergamotene, nerol, neral, cedrol, cedrene, thujopsene, methyl thujate, and thujic acid.

The anhydrous hair treatment composition optionally includes one or more emollients, for example, one or more fatty ethers, fatty esters, fatty carbonates, and/or propylene glycol esters of fatty acids.

Nonlimiting examples of fatty esters include coco-caprylate/caprate, coco-caprylate, coco-caprate, jojoba esters, isopropyl jojobate, ethyl macadamiate, isoamyl laurate, heptyl undecylenate, methylheptyl isostearate, isostearyl isostearate, glyceryl ricinoleate, isostearyl palmitate, myristyl myristate, octyldodecyl myristate, octyldodecyl hydroxystearate, butyl myristate, ethylhexyl cocoate, ethylhexyl palmitate, ethylhexyl stearate, butyl stearate, decyl oleate, isocetyl behenate, isocetyl myristate, isocetyl palmitate, isocetyl stearate, isodecyl oleate, isopropyl isostearate, isopropyl myristate, isopropyl palmitate, oleyl oleate, propylene glycol laurate, octydodecyl erucate, C12-C13 alkyl lactate, C12-C15 alkyl lactate, isostearyl lactate, glycereth-5 lactate, lauryl lactate, myristyl lactate, oleyl lactate, laureth-2 benzoate, C12-C15 alkyl benzoate, C12-C15 pareth-3 benzoate, dipropylene glycol benzoate, isodecyl salicylate, C12-C15 alkyl salicylate, tridecyl salicylate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, laureth-2 ethylhexanoate, cetearyl ethylhexanoate, isodecyl neopentanoate, isostearyl neopentanoate, myristyl neopentanoate, isostearyl behenate, octyldodecyl neopentanoate, and tridecyl neopentanoate.

Nonlimiting examples of fatty ethers include dicaprylyl ether, didecyl ether, dimyristyl ether, distearyl ether, and dilauryl ether.

Nonlimiting examples of fatty carbonates includes C14-15 dialkyl carbonate, dicaprylyl carbonate, diethyl carbonate, dihexyl carbonate, diethylhexyl carbonate, dimethoxyphenyl phenyloxoethyl ethylcarbonate, dimethyl carbonate, dipropyl carbonate, dipropylheptyl carbonate, and dioctyl carbonate.

The anhydrous hair treatment composition optionally includes one or more polymeric oil thickening agents and/or one or more cationic conditioning polymers. Nonlimiting examples of polymeric oil thickening agents include VP/hexadecene copolymer, trimethylpentanediol/adipic acid/glycerin crosspolymer, ethylene/propylene/styrene copolymer, butylene/ethylene/styrene copolymer, VP/eicosene copolymer, bis-dioctadecylamide dimer dilinoleic acid/ethylenediamine copolymer, cetearyl dimethicone crosspolymer, hydrogenated poly(C6-20 olefin), HDI/trimethylol hexyllactone crosspolymer, dimethicone/vinyl dimethicone crosspolymer, and vinyl dimethicone/methicone silsesquioxane crosspolymer. In various embodiments, preferred polymeric oil thickening agents include hydrogenated poly(C6-20 olefin), and HDI/trimethylol hexyllactone crosspolymer.

The anhydrous hair treatment composition optionally includes one or more miscellaneous ingredients. Nonlimiting examples of miscellaneous ingredients include preservatives, fragrances, chelating agents, antioxidants, flavonoids, vitamins, botanical extracts, UV filtering agents, proteins, protein hydrolysates, protein isolates, fillers, and composition colorants.

The anhydrous hair treatment composition is useful in methods for treating hair, for example, methods for conditioning hair, methods for smoothing hair, and/or methods for styling hair. The anhydrous hair treatment composition can be formulated and used as a leave-in product or formulated and used as a rinse-out product. Preferably, however, the anhydrous hair treatment composition is a leave-on product, which is applied to hair, for example, wet or damp hair. After application to the wet or damp hair, the anhydrous hair treatment composition is allowed to dry naturally or can be forcibly dried, for example, with a blow dryer. The anhydrous hair treatment composition is used in methods for conditioning hair, moisturizing hair, controlling frizz, imparting shine, and improving the look and feel of the hair.

### DETAILED DESCRIPTION OF THE DISCLOSURE

The present case pertains to an anhydrous hair treatment composition that provides softness, moisturization, and conditioning benefits to hair, and to methods for treating the hair with the anhydrous hair treatment composition. The anhydrous hair treatment composition is typically used as a leave-in product but is also useful as a rinse-out product. The anhydrous hair treatment composition moisturizes hair, reduces or prevents frizz, improves curl definition, and imparts softness and shine to the hair. The anhydrous hair treatment composition typically includes:
(a) about 10 to about 95 wt. % of one or more vegetable oils, wherein one of the one or more vegetable oils is castor oil in an amount of at least 10 wt. %;
(b) about 0.1 to about 10 wt. % of one or more cationic surfactants;
(c) about 0.1 to about 10 wt. % of one or more fatty alcohols; and
(d) optionally, about 0.1 to about 5 wt. % of one or more sensorially active agents;
   wherein all weight percentages are based on a total weight of the composition.

Preferably, the anhydrous hair treatment composition is free or essentially free from mono-alcohols having from 1 to 6 carbon atoms and free or essentially free from polyhydric alcohols having from 1 to 6 carbon atoms. Furthermore, in various embodiments, the anhydrous hair treatment composition is additionally, or alternatively, free or essentially free from esters of alpha hydroxy acids. The anhydrous hair treatment composition may additionally, or alternatively, be essentially free from acetal and ketals having 10 carbon atoms or less. A nonlimiting example of a ketal having 10 carbon atoms or less is isopropylidene glycerol. In a preferred embodiment, the anhydrous hair treatment composition is free or essentially free from isopropylidene glycerol.

### (a) Vegetable Oils

The anhydrous hair treatment composition includes one or more vegetable oils, provided that one of the one or more vegetable oils is castor oil (also known as “castor seed oil”) in an amount of at least 10 wt. %, based on the total weight of the anhydrous hair treatment composition. For purposes of the instant disclosure vegetable oils are oils from plants that are liquid at room temperature (25° C., 1 atm) or can be solubilized with other vegetable oils and exist as a liquid at room temperature. For example, coconut oil can be a solid a room temperature but if solubilized in castor oil, it exists as a liquid at room temperature. Vegetable oils come from a variety of plant sources, such as walnuts, almonds, grape seeds, olives, and avocados. For example, coconut oil is extracted from the pulp, olive oil is extracted from the fruit, and jojoba oil is extracted from the seed. Vegetable oils are typically obtained by pressing, which can be either cold or hot. Cold pressing involves pressing the plant to extract the oil, without raising the temperature above 40° C. Hot pressing involves heating the plant, which increases the oil yield but can alter its quality.

For purposes of the instant disclosure, the term “vegetable oil” does not encompass essential oils and the term “essential oil” does not encompass vegetable oils, even though essential oils are obtained from plants. In cases where overlap might exist such that a particular oil might construed as both a vegetable oil and an essential oil, a single oil cannot simultaneously function as the vegetable oil and the essential oil in the anhydrous hair treatment composition.

The total amount of the one or more vegetable oils in the anhydrous hair treatment composition will vary but is typically about 10 to about 95 wt. %, based on the total weight of the anhydrous hair treatment compositions. In further embodiments, the anhydrous hair treatment composition includes about 10 to about 90 wt. %, about 10 to about 80 wt. %, about 10 to about 70 wt. %, about 10 to about 65 wt. %, about 20 to about 95 wt. %, about 20 to about 90 wt. %, about 20 to about 80 wt. %, about 20 to about 70 wt. %, about 20 to about 65 wt. %, about 35 to about 95 wt. %, about 35 to about 90 wt. %, about 35 to about 80 wt. %, about 35 to about 70 wt. %, about 35 to about 65 wt. %, about 35 to about 55 wt. %, about 35 to about 50 wt. %, about 35 to about 45 wt. %, about 40 to about 75 wt. %, about 40 to about 65 wt. %, about 40 to about 55 wt. %, about 45 to about 75 wt. %, about 45 to about 65 wt. %, about 45 to about 55 wt. %, about 50 to about 75 wt. %, about 50 to about 70 wt. %, about 50 to about 65 wt. %, about 50 to about 60 wt. %, about 55 to about 75 wt. %, about 55 to about 70 wt. %, or about 55 to about 65 wt. % of the one or more vegetable oils, based on the total weight of the anhydrous hair treatment composition. Preferably, the anhydrous composition includes about 10 to about 70 wt. %, more preferably about 45 to about 65 wt. %, and even more preferably, about 50 to about 65 wt. % of the one or more vegetable oils, based on the total weight of the hair treatment composition.

(a)(i) Castor Oil

Castor oil is a vegetable oil pressed from castor beans. It is a colorless or pale-yellow liquid having a boiling point of about 313° C. (595° F.) and density of about 0.961 g/cm3. It includes a mixture of triglycerides in which about 90% of fatty acids are ricinoleates. Oleic acid and linoleic acid are other significant components. Thus, castor oil is well known as a source of ricinoleic acid, a monounsaturated, 18-carbon fatty acid. Among fatty acids, ricinoleic acid is unusual in that it has a hydroxyl functional group on the twelfth carbon atom. This functional group causes ricinoleic acid (and castor oil) to be more polar than many other oils.

The anhydrous hair treatment composition typically includes a substantial amount of castor oil, preferably at least 10 wt. % or more castor oil, based on a total weight of the anhydrous hair treatment composition. For example, the anhydrous hair treatment composition may include about 10 to about 95 wt. % of castor oil, based on the total weight of the anhydrous hair treatment composition. In further embodiments, the anhydrous hair treatment composition includes about 10 to about 55 wt. %, about 10 to about 50 wt. %, about 10 to about 45 wt. %, about 10 to about 40 wt. %, about 10 to about 37.5 wt. %, about 15 to about 55 wt. %, about 15 to about 50 wt. %, about 15 to about 45 wt. %, about 15 to about 40 wt. %, about 15 to about 37.5 wt. %, about 20 to about 55 wt. %, about 20 to about 50 wt. %, about 20 to about 45 wt. %, about 20 to about 40 wt. %, about 20 to about 37.5 wt. %, about 25 to about 55 wt. %, about 25 to about 50 wt. %, about 25 to about 45 wt. %, about 25 to about 40 wt. %, about 25 to about 37.5 wt. %, about 30 to about 60 wt. %, about 30 to about 55 wt. %, about 30 to about 50 wt. %, about 30 to about 45 wt. %, about 30 to about 40 wt. %, about 30 to about 37.5 wt. %, about 35 to about to about 55 wt. %, about 35 to about 50 wt. %, about 35 to about 45 wt. %, or about 35 to about 40 wt. % of castor oil, based on the total weight of the anhydrous hair treatment composition. The anhydrous hair treatment composition preferably includes about 10 to about 80 wt. %, more preferably about 15 to about 60 wt. %, and even more preferably, about 25 to about 50 wt. % of castor oil, based on the total weight of the anhydrous hair treatment composition.

(a)(ii) Additional Vegetable Oils (Other than Castor Oil)

As mentioned above, the anhydrous hair treatment composition preferably includes one or more additional vegetable oils (other than castor oil), i.e., the anhydrous hair treatment composition includes two or more (or a plurality of) vegetable oils, provided that one of the two or more (or plurality of) vegetable oils is castor oil. Nonlimiting examples of additional vegetable oils include coconut oil, arachis oil, amaranth seed oil, apricot oil, argan oil, avocado oil, babassu oil, cottonseed oil, borage seed oil, camelina oil, camellia oil, safflower oil, peanut oil, pomegranate seed oil, grapefruit seed oil, hemp oil, hazelnut oil, elderberry seed oil, currant seed oil, jojoba oil, linseed oil, macadamia nut oil, corn oil, almond oil, manila oil, evening primrose oil, olive oil, palm oil, palm kernel oil, brazil nut oil, pecan nut oil, peach kernel oil, rapeseed oil, rosehip oil, sea buckthorn fruit oil, sea buckthorn kernel oil, sesame oil, soybean oil, sunflower oil, grapeseed oil, walnut oil, wild rose oil, and wheat germ oil. In a preferred embodiment, the anhydrous hair treatment composition includes castor oil and one or more additional vegetable oils selected from coconut oil, soybean oil, avocado oil, arachis oil, apricot oil, argan oil, avocado oil, babassu oil, cottonseed oil, camelina oil, camellia oil, safflower oil, and peanut oil, preferably coconut oil and/or soybean oil.

In a preferred embodiment, the anhydrous hair treatment composition includes castor oil and one or more additional vegetable oils selected from coconut oil, and soybean oil. In another preferred embodiment, the hair treatment composition includes castor oil and coconut oil. In yet another preferred embodiment, the anhydrous hair treatment composition includes castor oil, coconut oil, and soybean oil, and optionally, one or more further vegetable oils (other than castor oil, coconut oil, and soybean oil).

The total amount of the one or more additional vegetable oils (other than castor oil) will vary but is typically about 1 to about 45 wt. %, based on the total weight of the anhydrous hair treatment composition. In further embodiments, the anhydrous hair treatment composition includes about 1 to about 35 wt. %, about 1 to about 25 wt. %, about 1 to about 20 wt. %, about 1 to about 15 wt. %, about 5 to about 45 wt. %, about 5 to about 35 wt. %, about 5 to about 25 wt. %, about 5 to about 20 wt. %, about 5 to about 15 wt. %, about 10 to about 45 wt. %, about 10 to about 35 wt. %, about 10 to about 25 wt. %, about 10 to about 20 wt. %, about 10 to about 15 wt. %, about 15 to about 45 wt. %, about 15 to about 35 wt. %, about 15 to about 25 wt. %, about 20 to about 45 wt. %, about 20 to about 35 wt. %, about 20 to about 20 wt. %, or about 20 to about 25 wt. % of the one or more additional vegetable oils (other than castor oil), based on the total weight of the anhydrous hair treatment composition. In preferred embodiments, the anhydrous hair treatment composition includes about 1 to about 35 wt. %, more preferably about 5 to about 30 wt. %, and even more preferably about 10 to about 25 wt. % of the one or more additional vegetable oils (other than castor oil), based on the total weight of the anhydrous hair treatment composition.

(a)(iii) Coconut Oil

In various embodiments, one of the one or more additional vegetable oils (other than castor oil) is coconut oil. Coconut oil is extracted from the kernel of coconuts harvested from the coconut palm and is high in saturated fats. Coconut oil provides a variety of benefits to hair. For example, in hydrates dry and damaged hair, reduces frizz, and protect hair against styling damage. It is safe to use on most hair types. Itis packed with fatty acids (especially lauric acid) that support hair health. Therefore, in addition to castor oil, it can be beneficial to also include coconut oil in the anhydrous hair treatment composition.

The total amount of coconut oil in hair treatment composition, if present, will vary but is typically about 1 to about 20 wt. %, based on the total weight of the anhydrous hair treatment composition. In further embodiments, the anhydrous hair treatment composition includes about 1 to about 18 wt. %, about 1 to about 15 wt. %, about 1 to about 12 wt. %, about 1 to about 10 wt. %, about 2 to about 20 wt. %, about 2 to about 18 wt. %, about to 2 to about 15 wt. %, about 2 to about 12 wt. %, about 2 to about 10 wt. %, about 5 to about 20 wt. %, about 5 to about 18 wt. %, about 5 to about 15 wt. %, about 5 to about 12 wt. %, about 8 to about 20 wt. %, about 8 to about 18 wt. %, about 8 to about 15 wt. %, or about 8 to about 12 wt. %, based on the total weight of the anhydrous hair treatment composition. Preferably, the anhydrous hair treatment composition includes about 2 to about 18 wt. %, more preferably about 5 to about 15 wt. %, and even more preferably 6 to about 14 wt. % of coconut oil, based on the total weight of the hair treatment compositions.

In a preferred embodiment, the anhydrous composition includes castor oil, coconut oil, and optionally one or more additional vegetable oils. In particular, the anhydrous hair treatment composition may include:
(a)(i) about 5 to about 75 wt. %, preferably about 30 to about 50 wt. %, more preferably about 30 to about 45 wt. %, and even more preferably about 35 to about 45 wt. % of castor oil;
(a)(ii) about 1 to about 25 wt. %, preferably about 2 to about 18 wt. %, more preferably about 5 to about 15 wt. %, and even more preferably about 6 to about 14 wt. % of coconut oil; and
(a)(iii) optionally, about 1 to about 20 wt. %, preferably about 2 to about 18 wt. %, more preferably about 5 to about 15 wt. %, and even more preferably about 6 to about 14 wt. % of one or more further vegetable oils (other than castor oil and coconut oil), for example, one or more further vegetable oils selected from soybean oil, avocado oil, arachis oil, apricot oil, argan oil, avocado oil, babassu oil, cottonseed oil, camelina oil, camellia oil, safflower oil, and peanut oil, preferably coconut oil and/or soybean oil.

### (b) Cationic Surfactants

For purposes of the present disclosure, the term “cationic surfactant” means a surfactant that bears one or more permanent positive charges or bears one or more functional groups that are cationizable, for example, based on pH. Quaternary ammonium surfactants have one or more permanent positive charges. Amidoamine surfactants are cationizable.

Nonlimiting examples of cationic surfactants include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethylamine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, and palmitamidopropyl dimethylamine.

In various embodiments, one or more cationic surfactants are preferably selected from quaternary ammonium compounds. Nonlimiting examples of quaternary ammonium compounds include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, and tricetylmonium chloride.

In various embodiments, one or more cationic surfactants are preferably selected from amidoamine surfactants. Nonlimiting examples of amidoamine surfactants include oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethylamine, behenamidoethyldimethylamine, arachidamidopropyldimethylamine, arachidamidopropyidiethylamine, and arachidamidoethyidiethylamine, arachidamidoethyidimethylamine.

In further embodiments, the one or more cationic surfactants are preferably selected from cetrimonium chloride, behentrimonium chloride, behentrimonium methosulfate, and stearamidopropyl dimethylamine.

In a preferred embodiment, at least one of the one or more cationic surfactants is an amidoamine surfactants. In further embodiments, all of the one or more cationic surfactants are selected from amidoamine surfactants. Nonlimiting example of preferably amidoamine surfactants include oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicamidopropyl dimethylamine, olivamidopropyl dimethylamine, and stearamidoethyldiethylamine.

The total amount of the one or more cationic surfactants in the anhydrous hair treatment composition will vary but is typically from about 0.1 to about 10 wt. %, based on the total weight of the anhydrous hair treatment composition. In further embodiments, the anhydrous hair treatment composition includes about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more cationic surfactants, based on the total weight of the anhydrous hair treatment composition. Preferably, the anhydrous hair treatment composition includes about 0.1 to about 10 wt. %, more preferably about 0.5 to about 8 wt. %, even more preferably about 0.8 to about 5 wt. %, and even more preferably about 1 to about 3 wt. % of one or more cationic surfactants, based on the total weight of the anhydrous hair treatment composition.

### (c) Fatty Alcohols

The term “fatty alcohol” means an alcohol comprising at least one hydroxyl group (OH), and comprising at least 8 carbon atoms, and which is neither oxyalkylenated (in particular neither oxyethylenated nor oxypropylenated) nor glycerolated. The fatty alcohols can be represented by: R—OH, wherein R denotes a saturated (alkyl) or unsaturated (alkenyl) group, linear or branched, comprising from 8 to 40 carbon atoms, preferably 10 to 30 carbon atoms, more preferably 12 to 24 carbon atoms, and even more preferably 14 to 22 carbon atoms. The fatty alcohols may be liquid or solid.

The total amount of the one or more fatty alcohols in the anhydrous hair treatment composition will vary but is typically from about 0.1 to about 15 wt. %, based on the total weight of the anhydrous hair treatment composition. In further embodiments, the anhydrous hair treatment composition includes about 0.1 to about 12 wt. %, about 0.1 to about 10 wt. %, about 0.1 to about 8 wt. %, about 0.5 to about 15 wt. %, about 0.5 to about 12 wt. %, about 0.5 to about 10 wt. %, about 0.5 to about 8 wt. %, about 1 to about 15 wt. %, about 1 to about 12 wt. %, about 1 to about 10 wt. %, about 1 to about 8 wt. %, about 2 to about 15 wt. %, about 2 to about 12 wt. %, about 2 to about 10 wt. %, about 2 to about 8 wt. %, about 4 to about 15 wt. %, about 4 to about 12 wt. %, about 4 to about 10 wt. %, or about 4 to about 8 wt. % of one of more fatty alcohols, based on the total weight of the anhydrous hair treatment composition. Preferably, the anhydrous hair treatment composition includes about 0.1 to about 15 wt. %, more preferably about 1 to about 12 wt. %, even more preferably about 2 to about 10 wt. %, and even more preferably about 4 to about 8 wt. % of one or more fatty alcohols, based on the total weight of the anhydrous hair treatment composition.

In various embodiments, it is preferable for the anhydrous hair treatment composition to include at least one solid fatty alcohol. The term “solid fatty alcohol” means a fatty alcohol having a melting point greater than 27° C. at atmospheric pressure (1.013×10⁵ Pa). For the purposes of the present disclosure, the melting point corresponds to the temperature of the most endothermic peak observed on thermal analysis (differential scanning calorimetry or DSC) as described in the standard ISO 11357-3; 1999. The melting point may be measured using a differential scanning calorimeter (DSC), for example the calorimeter sold under the name “MDSC 2920” by the company TA Instruments.

Nonlimiting examples of solid fatty alcohols include myristyl alcohol (1-tetradecanol), cetyl alcohol (1-hexadecanol), stearyl alcohol (1-octadecanol), arachidyl alcohol (1-eicosanol), behenyl alcohol (1-docosanol), lignoceryl alcohol (1-tetracosanol), ceryl alcohol (1-hexacosanol), montanyl alcohol (1-octacosanol), and myricyl alcohol (1-triacontanol). In a preferred embodiment, the one or more solid fatty alcohols are selected from myristyl alcohol, cetyl alcohol, stearyl alcohol, behenyl alcohol and mixtures thereof, such as cetylstearyl alcohol, and cetearyl alcohol. Preferably, the cosmetic composition includes one or more solid fatty alcohols chosen from cetyl alcohol, myristyl alcohol, stearyl alcohol, and behenyl alcohol, preferably the solid fatty alcohol is cetyl alcohol, myristyl alcohol, behenyl alcohol, and cetearyl alcohol.

The total amount of the one or more solid fatty alcohols, if present, in the anhydrous hair treatment composition will vary but is typically from about 0.1 to about 10 wt. %, based on the total weight of the anhydrous hair treatment composition. In further embodiments, the anhydrous hair treatment composition includes about 0.1 to about 8 wt. %, about 0.1 to about 6 wt. %, about 0.2 to about 10 wt. %, about 0.2 to about 8 wt. %, about 0.2 to about 6 wt. %, about 0.5 to about 10 wt. %, about 0.5 to about 8 wt. %, about 0.5 to about 6 wt. %, about 1 to about 10 wt. %, about 1 to about 8 wt. %, about 1 to about 6 wt. %, about 2 to about 10 wt. %, about 2 to about 8 wt. %, about 2 to about 6 wt. %, about 3 to about 10 wt. %, about 3 to about 8 wt. %, or about 3 to about 6 wt. %, based on the total weight of the anhydrous hair treatment composition. Preferably, the anhydrous hair treatment composition includes from about 0.1 to about 10 wt. %, more preferably about 1 to about 8 wt. %, more preferably about 2 to about 8 wt. %, and even more preferably about 2 to about 6 wt. % of one or more solid fatty alcohols.

In some instances, it is preferably if the anhydrous hair treatment composition includes at least one liquid fatty alcohol. The term “liquid fatty alcohol” means a fatty alcohol having a melting point of less than or equal to 27° C. at atmospheric pressure (1.013×10⁵ Pa). For the purposes of the present disclosure, the melting point corresponds to the temperature of the most endothermic peak observed on thermal analysis (differential scanning calorimetry or DSC) as described in the standard ISO 11357-3; 1999. The melting point may be measured using a differential scanning calorimeter (DSC), for example the calorimeter sold under the name “MDSC 2920” by the company TA Instruments.

The liquid fatty alcohols, in particular those containing C10-C34, preferably have branched carbon chains and/or have one or more, preferably 1 to 3 double bonds. They are preferably branched and/or unsaturated (C═C double bond) and contain from 12 to 40 carbon atoms.

The liquid fatty alcohols may be represented by: R—OH, wherein R denotes a C12-C24 branched alkyl group or an alkenyl group (comprising at least one C12-C24 double bond C═C), R being optionally substituted by a or more hydroxy groups. Preferably, the liquid fatty alcohol is a branched saturated alcohol. Preferably, R does not contain a hydroxyl group. These include oleic alcohol, oleyl alcohol, linoleic alcohol, linolenic alcohol, isocetyl alcohol, isostearyl alcohol, 2-octyl-1-dodecanol, 2-butyloctanol, 2-hexyl-1-decanol, 2-decyl-1-tetradecanol, and 2-tetradecyl-1-cetanol. Preferably, the liquid fatty alcohol is 2-octyl-1-dodecanol. Preferably, one or more liquid fatty alcohols are selected from 2-octyldodecanol, isostearyl alcohol, 2-hexyldecanol, 2-butyloctanol, 2-undecylpentadecanol, linoleyl alcohol, linolenyl alcohol, undecylenyl alcohol, Isostearyl alcohol, lanolin alcohol, oleyl alcohol, hexadecyl alcohol, octyldodecanol alcohol, linoleyl alcohol, linolenyl alcohol, lauryl alcohol, and arachidyl alcohol.

The total amount of the one or more liquid fatty alcohols, if present, in the anhydrous hair treatment composition will vary but is typically from about 0.1 to about 10 wt. %, based on the total weight of the anhydrous hair treatment composition. In further embodiments, the anhydrous hair treatment composition includes about 0.1 to about 8 wt. %, about 0.1 to about 6 wt. %, about 0.1 to about 5 wt. %, about 0.1 to about 4 wt. %, about 0.5 to about 10 wt. %, about 0.5 to about 8 wt. %, about 0.5 to about 6 wt. %, about 0.5 to about 5 wt. %, about 0.5 to about 4 wt. %, about 1 to about 10 wt. %, about 1 to about 8 wt. %, about 1 to about 6 wt. %, about 1 to about 5 wt. %, or about 1 to about 4 wt. % of the one or more liquid fatty alcohols, based on the total weight of the anhydrous hair treatment composition. Preferably, the anhydrous hair treatment composition includes about 0.1 to about 10 wt. %, preferably about 0.5 to about 8 wt. %, more preferably about 0.5 to about 6 wt. %, and even more preferably about 1 to about 4 wt. % of the one or more liquid fatty alcohols, based on the total weight of the anhydrous hair treatment composition.

In a preferred embodiment, the anhydrous hair treatment composition includes a combination of one or more solid fatty alcohols and one or more liquid fatty alcohols. For example, the combination of the combination of the one or more solid fatty alcohols and the one or more liquid fatty alcohols comprises, consists essentially of, or consists of:
(i) about 0.1 to about 12 wt. %, preferably about 0.5 to about 10 wt. %, more preferably about 1 to about 8 wt. %, and even more preferably about 2 to about 6 wt. % of one or more solid fatty alcohols are selected from stearyl alcohol, cetyl alcohol, cetearyl alcohol, myristyl alcohol, and behenyl alcohol; and
(ii) about 0.1 to about 10 wt. %, preferably about 0.2 to about 8 wt. %, more preferably about 0.5 to about 6 wt. %, and even more preferably about 1 to about 5 wt. % of one or more liquid fatty alcohols selected from isostearyl alcohol, oleyl alcohol, octyldodecanol, linoleyl alcohol, linolenyl alcohol, lauryl alcohol, caprylic alcohol, and capric alcohol.

### Ratio of Solid Fatty Alcohols to Liquid Fatty Alcohols

In instances where the anhydrous hair treatment composition includes a combination of one or more solid fatty alcohols and one or more liquid fatty alcohols, the weight of the one or more solid fatty alcohols to the one or more liquid fatty alcohols will vary. Nonetheless, the weight ratio is typically from about 10:1 to about 1:5 (solid fatty alcohols:liquid fatty alcohols). In further embodiments, the weight ratio of the one or more solid fatty alcohols to the one or more liquid fatty alcohols is from about 10:1 to about 1:2, about 8:1 to about 1:2, about 6:1 to about 1:2, about 5:1 to about 1:2, about 10:1 to about 1:1, about 8:1 to about 1:1, about 6:1 to about 1:1, about 5:1 to about 1:1, about 10:1 to about 1.5:1, about 8:1 to about 1.5:1, about 6:1 to about 1.5:1, about 5:1 to about 1.5:1, about 10:1 to about 1.8:1, about 8:1 to about 1.8:1, about 6:1 to about 1.8:1, or about 5:1 to about 1.8:1. In a further preferred embodiment, the total amount of the one or more solid fatty alcohol is greater than or equal to the total amount of the one or more liquid fatty alcohols. Preferably, the weight of the one or more solid fatty alcohols to the one or more liquid fatty alcohols is from about 10:1 to about 1:2, more preferably from about 8:1 to about 1:1, and even more preferably from about 6:1 to about 1.2:1.

### Ratio of (b) to (c)

The one or more cationic surfactants of (b) and the one or more fatty alcohols of (c) are in a weight ratio that will vary. Nonetheless, the weight ratio of (b) to (c) can be about 1:12 to about 2:1 ((b):(c)). In further embodiments, the weight ratio of (b) to (c) is about 1:12 to about 2:1, about 1:12 to about 1:1, about 1:12 to about 1:2, about 1:12 to about 1:4, about 1:12 to about 1:5, about 1:10 to about 2:1, about 1:10 to about 1:1, about 1:10 to about 1:2, about 1:10 to about 1:3, about 1:10 to about 1:4, about 1:10 to about 1:5, about 1:8 to about 2:1, about 1:8 to about 1:1, about 1:8 to about 2:1, about 1:8 to about 3:1, about 1:8 to about 1:4, or about 1:8 to about 1:5, about 1:7 to about 2:1, about 1:7 to about 1:1, about 1:7 to about 1:2, or about 1:7 to about 1:4. Preferably the weight ratio of (b) to (c) is about 1:12 to about 1:1, preferably about 1:10 to about 1:2, more preferably about 1:8 to about 1:4.

### (d) Sensorially Active Agents

A sensorially active agent is a substance or compound that imparts or causes an individual (a human) to experience a sensation, for example, a sensation to the skin, a pleasant odor, and/or a pleasant taste. Nonlimiting examples include essential oils, cooling agents, warming agents, terpenes, and terpenoids. Overlap exists between essential oils, cooling agents, warming agents, and terpenes and/or terpenoids. For example, menthol can be provided as an essential oil, is a cooling agent, and is a terpene. In instances where overlap exists, a single substance or compound will be construed as one of essential oil, cooling agent, warming agent, terpene and/or terpenoid. A single substance or compound does not simultaneous act as more than one of an essential oil, cooling agent, warming agent, or terpene and/or terpenoid. For example, when menthol is included in an anhydrous hair treatment composition, menthol is construed as an essential oil if included or added as an essential oil or menthol services as a cooling agent or as a terpene.

The total amount of the one or more sensorially active agents in the anhydrous hair treatment composition, if present, will vary but is typically about 0.05 to about 8 wt. %, based on the total weight of the anhydrous hair treatment composition. In further embodiments, the anhydrous hair treatment composition includes about 0.05 to about 5 wt. %, about 0.5 to about 4 wt. %, about 0.5 to about 3 wt. %, about 0.1 to about 5 wt. %, about 0.1 to about 4 wt. %, about 0.1 to about 3 wt. %, about 0.5 to about 5 wt. %, about 0.5 to about 4 wt. %, about 0.5 to about 3 wt. %, about 1 to about 5 wt. %, about 1 to about 4 wt. %, or about 1 to about 3 wt. % of the one or more cooling agents, based on the total weight of the anhydrous hair treatment composition. Preferably, the anhydrous hair treatment composition includes about 0.05 to about 5 wt. %, more preferably about 0.1 to about 4 wt. %, and even more preferably about 0.5 to about 3 wt. % of the one or more sensorially active agents, based on the total weight of the anhydrous hair treatment composition.

(d)(i) Essential Oils

The anhydrous hair treatment composition may include one or more essential oils. Essential oils are concentrated liquid extracts of aromatic plants, typically in the form of volatile oils containing compounds that give plants their characteristic odors and therapeutic properties. Distillation involves passing water vapor through the plant, causing the aromatic compounds to evaporate. The vapor is then cooled and condensed, separating the essential oil from the water. Essential oils come from various parts of plants, such as flowers, leaves, bark, roots, or fruit. For example, lavender essential oil is extracted from the flowers, lemon essential oil is extracted from the zest, and ginger essential oil is extracted from the rhizome. Essential oils have a unique composition of chemicals that can vary within the same plant species, or from plant to plant.

For purposes of the instant disclosure, the term “essential oil” is used in accordance with The European Pharmacopoeia; and essential oils can be identified and characterized according to Do et al., Proposal for a standardised method for the identification of essential oils by HPTLC, PHARMEUR. BIO. SCI. NOTES. 2021; 2021:157-166 (PMID: 34751647), which are incorporated herein by reference in their entirety. Essential oils are from plants and may therefore fall within the broad umbrella of vegetable oils but may be considered a specific and unique type of vegetable oil. For purposes of the instant disclosure, the term “vegetable oil” does not encompass essential oils and the term “essential oil” does not encompass vegetable oils, even though essential oils are obtained from plants. In cases where overlap might exist such that a particular oil might construed as both a vegetable oil and an essential oil, a single oil cannot simultaneously function as the vegetable oil and the essential oil in the anhydrous hair treatment composition.

Nonlimiting examples of essential oils include lavender oil, geranium oil, juniper berry oil, bay leaf oil, lemon oil, mandarin orange oil, orange oil, absinthe oil, African bluegrass oil, ajowan oil, anethi oil, angelica root oil, anise star oil, arborvitae wild oil, armoise mugwort oil, asafetida oil, Australian balm oil, basil oil, benzoin oil, bergamot calabrian oil, betel leaf oil, birch sweet oil, birch tar oil, black currant-seed oil, blood orange oil, buchu oil, cabreuva oil, cade oil, cajeput oil, calamus root oil, camphor oil, cananga oil, caraway oil, cardamom oil, carrotseed oil, cassia oil, cedarleaf oil cedarwood atles oil, cedarwood Chinese oil, cedarwood Himalayan oil, cedarwood texas oil, cedarwood Virginian oil, celery seed oil, chamomile German oil, chamomile moroc oil, chamomile roman oil, chaulmogra oil, chenopodium oil, chilly seed oil, cinnamon bark oil, cinnamon leaf oil, citral oil, citronelia Ceylon oil, clarysage oil, clementine oil, clove bud oil, clove leaf oil, cognac oil, copaiba balsam oil, coriander oil, costus root oil, coffee oil, cumin oil, curcuma oil, curry leaf oil, cypress Australian blue oil, cypress French cypriol oil, davana oil, dill seed oil, dill weed oil, elemi oil, emu oil, eucalyptus oil, fennel bitter oil, fenugreek oil, fir balsam oil, fir needle oil, flax seed organic oil, frankincense oil garlic oil, galbanum oil, ginger oil, ginger grass oil, gaia oil, grapefruit pink oil, grapefruit white oil, guaiacwood oil, helichrysum oil, horseradish oil, hydacheim oil, hyssop oil, jamarosa root oil, Himalayan wild oil, karanj seed oil, kanuka oil, katrafay oil, key lime oil, kunzea oil, kukuinut oil, lantana oil, laurel leaf oil, lemongrass oil, lemon eucalyptus oil, lemon myrtle oil, lemon 5 fold oil, lican oil, lime oil, Litsea cubeba oil, lovage leaf oil, mace oil, mandarin oil, manuka oil, marjoram oil, Melissa leaf oil, menthol liquid oil, mullein oil, myrrh oil, myrtle oil, neem oil, neroli oil, nerolina Australian oil, niaouli oil, noni oil, nutmeg oil, oat oil, onion oil, opoponax oil, orange bitter oil, orange sweet oil, origanum oil, orange 5 fold oil, palmarosa oil, parsley oil, patchouli oil, pennyroyal oil, pepper oil, peppermint oil, peru balsam oil, petitgrain oil, pimento leaf oil, pine long leaf oil, pine scotch oil, pine commercial oil, pomegranate oil, pomouwood oil, ravensara wild oil, rosalina Australian oil, rose geranium oil, rosemary oil, rosewood oil, rue oil, sage Dalmatian oil, sandalwood oil, savine oil, savory winter oil, sea buckthorn oil, spearmint oil, spikenard oil, spruce oil, sugandh kokila oil, sumac oil, tagetes oil, tangerine oil, tansy blue oil, tarragon oil, tangerine 5 fold oil, tea tree oil, tea tree lemon oil, thyme oil, tolu balsam oil, turmeric oil, valerian root oil, vanilla 10 fold oil, vetiver oil, wintergreen oil, wild verbena oil, wormwood oil, yarrow oil, and ylang ylang oil.

In a preferred embodiment, the anhydrous hair treatment composition includes at least one essential oil selected from lavender, peppermint, rosemary, tea tree, Roman chamomile, bergamot, jasmine, Ylang-ylang, eucalyptus, geranium, patchouli, lemon, frankincense, lemongrass, cedarwood, and grapefruit.

The total amount of the one or more essential oils in the anhydrous hair treatment composition will vary but is typically about 0.01 to about 5 wt. %, based on the total weight of the anhydrous hair treatment composition. In further embodiments, the anhydrous hair treatment composition includes about 0.01 to about 4 wt. %, about 0.01 to about 3 wt. %, about 0.01 to about 2 wt. %, about 0.05 to about 5 wt. %, about 0.05 to about 4 wt. %, about 0.05 to about 3 wt. %, about 0.05 to about 2 wt. %, about 0.1 to about 5 wt. %, about 0.1 to about 4 wt. %, about 0.1 to about 3 wt. %, about 0.1 to about 2 wt. %, about 0.5 to about 5 wt. %, about 0.5 to about 4 wt. %, about 0.5 to about 3 wt. %, or about 0.5 to about 2 wt. % of the one or more essential oils, based on the total weight of the anhydrous hair treatment composition. Preferably, the anhydrous hair treatment composition includes about 0.05 to about 5 wt. %, more preferably about 0.1 to about 4 wt. %, and even more preferably about 0.5 to about 3 wt. % of the one or more essential oils, based on the total weight of the anhydrous hair treatment composition.

(d)(ii) Cooling Agents

The anhydrous hair treatment composition may include one or more cooling agents. As the name suggest, a cooling agent is a substance or compound that imparts or causes an individual (a human) to experience a cooling sensation. Nonlimiting examples of cooling agents include menthol, N-ethyl-p-menthyl-3-carboxiamide, cubebol, isopulegol, 3-(L-menthoxy) propane-1,2-diol, cis/trans-p-menthane-3,8-diol, 3-(L-menthoxy)-2-methylpropane-1,2-diol, 2-[2-(p-menthan-3-yloxy) ethoxy]ethanol, menthoxyethanol, (1R,2R,4R)-1-(2-hydroxy-4-methylcyclohexyl) ethenone, (1R,2R,5R)—N-ethyl-5-methyl-2-(prop-1-en-2-yl)-cyclohexanecarboxamide, N-(3-hydroxy-4-methoxyphenyl)-2-isopropyl-5,5-dimethyl-cyclohexanecarboxamide, N-[4-(cyanomethyl)phenyl]-2-isopropyl-5,5-dimethyl-cyclohexanecarboxamide, N-(2-Hydroxy-2-phenylethyl)-2-isopropyl-5,5-dimethyl-cyclohexane-1-carboxamide, L-menthol lactate, (−)-menthyl ethylene glycol carbonate, (−)-menthone 1,2-glycerol ketal, D/L-menthone 1,2-glycerol ketal, (−)-menthyl 1-propylene glycol carbonate, (−)-menthyl 2-propylene glycol carbonate, D/L-menthyl 1-propylene glycol carbonate, D/L-menthyl 2-propylene glycol carbonate, (1R,2S,5R)—N-(4-methoxyphenyl)-5-methyl-2-(1-methylethyl)cyclohexanecarboxamide, menthyl ethylamido oxalate, (E)-3-benzo[1,3]dioxol-5-yl-N,N-diphenyl-2-propenamide, 3,4-methylenedioxycinnamic acid, N,N-diphenylamide, N,N-dimethyl (−)-menthyl succinimide, (1R,2S,5R)—N-(4-(carbamoylmethyl)phenyl)-menthylcarboxamide, (−)-menthyl pyrrolidone carboxylate, L-phenylephrine p-menthane carboxamide, (1R,2S,5R)—N-(4-(cyanomethyl)-phenyl) menthylcarboxamide, (1R,2S,5R)—N-(2-(pyridin-2-yl)ethyl) menthylcarboxamide, 6-isopropyl-3,9-dimethyl-1,4-dioxaspiro[4.5]decan-2-one, (1R,2R,4S)-dihydroumbellulol, N-ethyl-p-menthane-3-carboxamide, 2-isopropyl-N,2,3-trimethylbutyramide, ethyl 3-(p-menthane-3-carboxamido)acetate, N-ethyl-2,2-diisopropylbutanamide, N-(1,1-dimethyl-2-hydroxyethyl)-2,2-dimethylbutanamide, N-cyclopropyl-5-methyl-2-isopropyl-cyclohexanecarbonecarboxamide, (−)-menthyl acetoacetate, (−)-menthyl succinate, (−)-menthyl (S)-3-hydroxybutyrate, (−)-menthyl glutarate, 1-[2-hydroxyphenyl]-4-[2-nitrophenyl]-1,2,3,6-tetrahydropyrimidine-2-one, di-(−)-menthyl glutarate, N-(2-hydroxyethyl)-2,3-dimethyl-2-isopropylbutanamide, and peppermint oil. In a preferred embodiment, the anhydrous hair treatment composition includes peppermint oil and menthol.

The total amount of the one or more cooling agents, if present, will vary but are typically about 0.01 to about 5 wt. %, based on the total weight of the anhydrous hair treatment composition. In further embodiments, the anhydrous hair treatment composition includes about 0.01 to about 4 wt. %, about 0.01 to about 3 wt. %, about 0.01 to about 2 wt. %, about 0.05 to about 5 wt. %, about 0.05 to about 4 wt. %, about 0.05 to about 3 wt. %, about 0.05 to about 2 wt. %, about 0.1 to about 5 wt. %, about 0.1 to about 4 wt. %, about 0.1 to about 3 wt. %, about 0.1 to about 2 wt. %, about 0.5 to about 5 wt. %, about 0.5 to about 4 wt. %, about 0.5 to about 3 wt. %, or about 0.5 to about 2 wt. % of the one or more cooling agents, based on the total weight of the anhydrous hair treatment composition. Preferably, the anhydrous hair treatment composition includes about 0.05 to about 5 wt. %, more preferably about 0.1 to about 4 wt. %, and even more preferably about 0.5 to about 3 wt. % of the one or more cooling agents, based on the total weight of the anhydrous hair treatment composition.

(d)(iii) Warming Agents

The anhydrous hair treatment composition may include one or more warming agents. As the name suggests, a “warming agent” is a substance or compound that imparts or causes an individual (a human) to experience a warming sensation. Nonlimiting examples of warming agents include vanillyl ethyl ether, vanillyl propyl ether, vanillin propylene glycol acetal, ethyl vanillin propylene glycol acetal, capsaicin, gingerol, vanillyl butyl ether, 4-(1-menthoxy-methyl)-2-phenyl-1,3-dioxolane, 4-(1-menthoxy-methyl)-2-(3′,4′-dihydroxy-phenyl)-1,3-dioxolane, 4-(1-menthoxy-methyl)-2-(2′-hydroxy-3′-methoxy-phenyl)-1,3-dioxolane, 4-(1-menthoxy-methyl)-2-(4′-methoxyphenyl)-1,3-dioxolane, 4-(1-menthoxy-methyl)-2-(3′,4′-methylenedioxy-phenyl)-1,3-dioxolane, black pepper extract, cinnamaldehyde, piperine, hot pepper oil, red pepper oleoresin, capsicum oleoresin, ginger oleoresin, nonyl acid vanillylamide, 4-(1-menthoxy-methyl)-2-(3′-methoxy-4′-hydroxyphenyl)-1,3-dioxolane, vanillin-1,2-hexylene glycol acetal, vanillin-1,2-butylene glycol acetal, vanillin-1-butoxyglycerol acetal, ethyl vanillin, ethyl vanillyl alcohol (3-ethoxy-4-hydroxybenzyl alcohol), ethyl homovanillate, and vanillyl isopropyl ether.

In a preferred embodiment, the anhydrous hair treatment composition includes at least one warming agent selected from vanillyl ethyl ether, vanillyl propyl ether, vanillin propylene glycol acetal, ethyl vanillin propylene glycol acetal, capsaicin, gingerol, vanillyl butyl ether, 4-(1-menthoxy-methyl)-2-phenyl-1,3-dioxolane, 4-(1-menthoxy-methyl)-2-(3′,4′-dihydroxy-phenyl)-1,3-dioxolane, 4-(1-menthoxy-methyl)-2-(2′-hydroxy-3′-methoxy-phenyl)-1,3-dioxolane, 4-(1-menthoxy-methyl)-2-(4′-methoxyphenyl)-1,3-dioxolane, 4-(1-menthoxy-methyl)-2-(3′,4′-methylenedioxy-phenyl)-1,3-dioxolane, black pepper extract, cinnamaldehyde, piperine, hot pepper oil, red pepper oleoresin, capsicum oleoresin, ginger oleoresin, nonyl acid vanillylamide, 4-(1-menthoxy-methyl)-2-(3′-methoxy-4′-hydroxyphenyl)-1,3-dioxolane, vanillin-1,2-hexylene glycol acetal, vanillin-1,2-butylene glycol acetal, vanillin-1-butoxyglycerol acetal, ethyl vanillin, ethyl vanillyl alcohol (3-ethoxy-4-hydroxybenzyl alcohol), and ethyl homovanillate, vanillyl isopropyl ether.

The total amount of the one or more warming agents, if present, will vary but are typically about 0.01 to about 5 wt. %, based on the total weight of the anhydrous hair treatment composition. In further embodiments, the anhydrous hair treatment composition includes about 0.01 to about 4 wt. %, about 0.01 to about 3 wt. %, about 0.01 to about 2 wt. %, about 0.05 to about 5 wt. %, about 0.05 to about 4 wt. %, about 0.05 to about 3 wt. %, about 0.05 to about 2 wt. %, about 0.1 to about 5 wt. %, about 0.1 to about 4 wt. %, about 0.1 to about 3 wt. %, about 0.1 to about 2 wt. %, about 0.5 to about 5 wt. %, about 0.5 to about 4 wt. %, about 0.5 to about 3 wt. %, or about 0.5 to about 2 wt. % of the one or more warming agents, based on the total weight of the anhydrous hair treatment composition. Preferably, the anhydrous hair treatment composition includes about 0.05 to about 5 wt. %, more preferably about 0.1 to about 4 wt. %, and even more preferably about 0.5 to about 3 wt. % of the one or more warming agents, based on the total weight of the anhydrous hair treatment composition.

(d)(vi) Terpenes/Terpenoids

The anhydrous hair treatment composition may include one or more terpenes and/or terpenoids. Terpenes are simple hydrocarbons, whereas terpenoids are a modified family of terpenes with various functional groups and an oxidized methyl group relocated or removed. Depending on the number of carbon units, terpenoids are classified as monoterpenes, sesquiterpenes, diterpenes, sesterpenes, and triterpenes. Nonlimiting examples of terpenes and terpenoids include menthol, myrcene, limonene, linalool, humulene, terpinolene, camphene, terpineol, beta-caryophyllene, 1,8-cineol, anisole, anethole, eugenol, thymol, borneol, citronellol, isomenthol, camphor, camphene, carvone, geraniol, menthone, pulegone, α-pinene, β-pinene, sabinene, α-terpinene, β-bisabolene, trans-α-bergamotene, nerol, neral, cedrol, cedrene, thujopsene, methyl thujate, and thujic acid.

The total amount of the one or more terpenes and/or terpenoids, if present, will vary but are typically about 0.01 to about 5 wt. %, based on the total weight of the anhydrous hair treatment composition. In further embodiments, the anhydrous hair treatment composition includes about 0.01 to about 4 wt. %, about 0.01 to about 3 wt. %, about 0.01 to about 2 wt. %, about 0.05 to about 5 wt. %, about 0.05 to about 4 wt. %, about 0.05 to about 3 wt. %, about 0.05 to about 2 wt. %, about 0.1 to about 5 wt. %, about 0.1 to about 4 wt. %, about 0.1 to about 3 wt. %, about 0.1 to about 2 wt. %, about 0.5 to about 5 wt. %, about 0.5 to about 4 wt. %, about 0.5 to about 3 wt. %, or about 0.5 to about 2 wt. % of the one or more terpenes and/or terpenoids, based on the total weight of the anhydrous hair treatment composition. Preferably, the anhydrous hair treatment composition includes about 0.05 to about 5 wt. %, more preferably about 0.1 to about 4 wt. %, and even more preferably about 0.5 to about 3 wt. % of the one or more terpenes and/or terpenoids, based on the total weight of the anhydrous hair treatment composition.

### (e) Emollients

The anhydrous hair treatment compositions may optionally include one or more emollients. Emollients are substances that help soothe, soften, and increase moisture levels in hair and skin. Nonlimiting examples of emollients include fatty ethers, fatty esters, and fatty carbonates, and caprylic/capric triglyceride. The anhydrous hair treatment composition includes one or more emollients, provided that one of the one or more emollients is caprylic/capric triglyceride, typically in an amount of at about 5 wt. % or more, based on the total weight of the anhydrous hair treatment composition.

The total amount of the one or more emollients, if present, will vary but is typically about 1 to about 55 wt. %, based on the total weight of the anhydrous hair treatment composition. In further embodiments, the anhydrous hair treatment composition includes about 1 to about 50 wt. %, about 1 to about 40 wt. %, about 1 to about 35 wt. %, about 5 to about 55 wt. %, about 5 to about 50 wt. %, about 5 to about 40 wt. %, about 5 to about 35 wt. %, about 10 to about 55 wt. %, about 10 to about 50 wt. %, about 10 to about 40 wt. %, about 10 to about 35 wt. %, about 20 to about 55 wt. %, about 20 to about 50 wt. %, about 20 to about 45 wt. %, about 20 to about 40 wt. %, about 25 to about 55 wt. %, about 25 to about 50 wt. %, about 25 to about 45 wt. %, about 25 to about 40 wt. %, about 30 to about 55 wt. %, about 30 to about 50 wt. %, about 30 to about 45 wt. %, about 30 to about 40 wt. %, about 35 to about 55 wt. %, about 35 to about 50 wt. %, about 35 to about 45 wt. %, or about 35 to about 40 wt. % of one or more emollients, based on the total weight of the anhydrous hair treatment composition. Preferably, the anhydrous hair treatment composition includes about 1 to about 55 wt. %, more preferably about 10 to about 50 wt. %, and even more preferably about 20 to about 45 wt. % of one or more emollients, based on the total weight of the anhydrous hair treatment composition.

Nonlimiting examples of fatty esters, fatty ethers, fatty carbonates, and caprylic/capric triglyceride are provided below.

(e)(i) Fatty Ethers

The anhydrous hair treatment composition may optionally include one or more fatty ethers. The fatty ethers may be monoalkyl fatty ethers or dialkyl fatty ethers ethers, and are optionally polyalkoxylated, for example polyethoxylated or polypropoxylated. At least one alkyl groups of the fatty ethers preferably have a carbon length of at least 4 carbons, preferably at least 6 carbons and may be saturated or unsaturated, linear or branched, and optionally substituted, for example, by one or more hydroxyl groups. For example, a least one of the alkyl groups may be saturated or unsaturated, linear or branched C₄-C₂₆, C₆-C₂₆, or C₆-C₂₄, optionally substituted, for example, with one or more hydroxyl groups. Preferably, the total number of carbon atoms in the fatty ether is greater than or equal to 10. In further embodiments, the total number of carbon atoms in the fatty ether is greater than 12, more preferably, greater than 14. In a preferred embodiment, the fatty ether is a dialkyl ether for formula: R₁OR₁, wherein R₁ represents a C₄-C₂₄-alkyl group, preferably a C₆-C₂₀-alkyl group, more preferably a C₆-C₁₈-alkyl group, and even more preferably a C₆-C₁₂-alkyl group. Nonlimiting examples of such dialkyl fatty ethers include dihexyl ether, dioctyl ether, diethylhexyl ether, dioctyl ether, dodecyl ether, and dicaprylyl ether

Nonlimiting examples of suitable polyoxyethylene fatty ethers includepolyoxyethylene cetyl/stearyl ether, polyoxyethylene cholesterol ether, polyoxyethylene laurate or dilaurate, polyoxyethylene stearate or distearate, and polyoxyethylene lauryl or stearyl ether, wherein the polyoxyethylene head group ranges from about 2 to about 100 groups. In certain embodiments, the polyoxyethylene fatty ethers include polyoxyethylene stearyl ether, polyoxyethylene myristyl ether, and polyoxyethylene lauryl ether having from about 3 to about 10 oxyethylene units.

In a preferred embodiment, at least one of the one or more fatty ethers is selected from a fatty dialkyl ether selected from dicaprylyl ether, didecyl ether, dimyristyl ether, distearyl ether, dilauryl ether, dihexyl ether, dioctyl ether, diethylhexyl ether, dioctyl ether, and dodecyl ether. In a preferred embodiment, at least one of the one or more fatty ethers is dicaprylyl ether.

The total amount of the one or more fatty ethers, if present, will vary but is typically about 1 to about 20 wt. %, based on the total weight of the anhydrous hair treatment composition. In further embodiments, the anhydrous hair treatment composition includes about 1 to about 18 wt. %, about 1 to about 15 wt. %, about 1 to about 12 wt. %, about 2 to about 20 wt. %, about 2 to about 18 wt. %, about 2 to about 15 wt. %, about 2 to about 12 wt. %, about 5 to about 20 wt. %, about 5 to about 18 wt. %, about 5 to about 15 wt. %, about 5 to about 12 wt. %, about 8 to about 20 wt. %, about 8 to about 18 wt. %, about 8 to about 15 wt. %, or about 8 to about 12 wt. % of the one or more fatty ethers, based on the total weight of the anhydrous hair treatment composition. Preferably, the anhydrous hair treatment composition includes about 2 to about 18 wt. %, more preferably about 4 to about 16 wt. %, and even more preferably about 6 to about 14 wt. % of the one or more fatty ethers, based on the total weight of the anhydrous hair treatment composition.

(e)(ii) Fatty Esters

The anhydrous hair treatment composition may optionally include one or more fatty esters. Nonlimiting examples of fatty esters include dicarboxylic acid fatty esters, monocarboxylic acid fatty esters, propylene glycol esters of fatty acids,

Nonlimiting examples of dicarboxylic acid fatty esters include those of the formula R₃OOCR₂COOR₃, which is obtainable from an esterification of a dicarboxylic acid (═HOOC—R₂COOH) and an mono alcohol (R₃—OH). R² represents a C₂₋₁₀-alkylene group, preferably a C₄₋₈-alkylene group, which optionally comprises at least one OH group. R₃ represents a C₂₋₁₄-alkyl group, preferably a C₂₋₈-alkyl group. The dicarboxylic acid may comprise at least one OH group. Nonlimiting examples of dicarboxylic acids comprising hydroxyl groups are tartaric acid, pentaric acid and 3-hydroxyglutaric acid. Nonlimiting examples of suitable alcohols (R₃—OH) include ethanol, propanol, iso-propanol, butanol, hexanol, octanol, 2-ethyhexanol, nonanol, iso-nonanol, decanol, iso-decanol, dodecanol and iso-dodecanol, preferably selected from the group consisting of ethanol, iso-propanol, butanol and 2-ethyhexanol. Preferred alcohols include ethanol, propanol, iso-propanol and butanol.

Nonlimiting examples of monocarboxylic acid fatty esters include aliphatic monoesters of the formula R₄COOR₅, which is obtainable from an esterification of a carboxylic acid (═R₄—COOH) and a mono alcohol (R₅—OH). R₄ represents a C₄₋₂₂-alkyl group, preferably a C₆₋₁₈-alkyl group. R⁵ represents a C₈₋₂₀-alkyl group, preferably a C₈₋₁₆-alkyl group, more preferably a C₁₀₋₁₆-alkyl group. Nonlimiting examples of suitable carboxylic acids include capronic acid, caprylic acid, 2-ethylhexanoic acid, capric acid, 3,5,5-trimethylhexanoic acid, isononanoic acid, lauric acid, myristic acid, palmitic acid, stearic acid and arachidonic acid. Nonlimiting examples of suitable alcohols (R₅—OH) include octanol, 2-ethyhexanol, nonanol, isononanol, decanol, iso-decanol, dodecanol isododecanol, tridecanol iso-tridecanol and cetearyl alcohol. Nonlimiting examples of fatty esters of the formula R₄COOR₅ include ethylhexyl cocoate, ethylhexyl palmitate, isotridecyl myristate, isotridecyl isononanoate, isodecyl ethylhexanoate, isodecyl isononanoate, isodecyl octanoate, isodecyl neopentanoate and cetearyl isononanoate.

Nonlimiting examples of propylene glycol fatty acid esters include propylene glycol esters of medium chain fatty acids (fatty acids having from 6 to 12 carbon atoms), such as propylene glycol dicaprylate/dicaprate, propylene glycol dipelargonate, and propylene glycol dilaurate. A preferred propylene glycol fatty acid ester is propylene glycol dicaprylate/dicaprate. The term “propylene glycol dicaprylate/dicaprate” is understood by those in the art to refer to a combination containing propylene glycol dicaprylate, propylene glycol dicaprylate-caprate, and propylene glycol dicaprate, which may vary in the ratio of these components. An example of a commercially available form of propylene glycol dicaprylate/dicaprate is CAPTEX® 200, available from the Abitec Corp. (Columbus, OH, USA).

In a preferred embodiment, the one or more fatty esters are selected from cetyl palmitate, cetyl stearate, cetyl esters, myristyl myristate, myristyl stearate, cetyl myristate, stearyl stearate, glyceryl stearate, propylene glycol dicaprylate/dicaprate, cetearyl ethylhexanoate, isopropyl isostearate, n-propyl myristate, isopropyl myristate, hexyl laurate, hexadecyl isostearate, hexydecyl laurate, hexyldecyl octanoate, n-propyl palmitate, and isopropyl palmitate.

The anhydrous hair treatment composition may preferably include one or more liquid fatty esters. The term “liquid fatty ester” means an ester that is liquid at room temperature and atmospheric pressure (25° C., 1 atm) and which comprises in its structure at least one hydrocarbon-based chain containing at least 6 carbon atoms. Preferably, it has a melting point of less than or equal to 10° C.

The liquid fatty esters include esters of monoalcohols or of polyols with monocarboxylic or polycarboxylic acids, at least one of the alcohols and/or acids comprising at least one hydrocarbon-based chain containing at least 6 carbon atoms. The liquid fatty esters include esters of a fatty acid (at least 6 carbon atoms) and of a monoalcohol, more particularly from esters of a fatty monoacid and of a monoalcohol. Preferably, at least one of the alcohols and/or acids is branched. Preferably, the alcohol and/or the acid are saturated, and preferably both are saturated. Moreover, the liquid fatty ester is preferably not oxyalkylenated.

The liquid fatty esters include those of formula R₆—COOR₇, in which: R₆ denotes a linear or branched, saturated or unsaturated, optionally mono- or polyhydroxylated hydrocarbon-based radical, containing from 5 to 31 carbon atoms, preferably containing from 7 to 21 carbon atoms, and more preferably from 8 to 20; and Rz denotes a linear or branched, saturated or unsaturated, optionally mono- or polyhydroxylated hydrocarbon-based radical, containing from 1 to 20 carbon atoms. Preferably, R₆ denotes a linear or branched alkyl (saturated) radical containing 6 to 22 carbon atoms, especially from 8 to 20 carbon atoms, and more preferably from 8 to 16 carbon atoms. Preferably, R₇ denotes a linear alkyl (saturated) radical containing 1 to 4 carbon atoms or a branched alkyl (saturated) radical containing from 2 to 20 carbon atoms, especially from 4 to 20 carbon atoms. More preferably, R₇ denotes a branched saturated alkyl radical containing from 4 to 12 carbon atoms.

Nonlimiting examples of preferred liquid fatty esters include ethyl laurate, butyl laurate, hexyl laurate, isohexyl laurate, isopropyl laurate, methyl myristate, ethyl myristate, butyl myristate, isobutyl myristate, isopropyl myristate, 2-octyldodecyl myristate, 2-ethylhexyl monococoate (or octyl monococoate), ethyl palmitate, isopropyl palmitate, isobutyl palmitate, 2-ethylhexyl palmitate (or octyl palmitate), butyl stearate, isopropyl stearate, isopropyl isostearate, isobutyl stearate, isocetyl stearate, isostearyl isostearate, isopropyl isostearate, 2-ethylhexyl stearate (or octyl stearate), 2-ethylhexyl hydroxystearate (or octyl hydroxystearate), decyl oleate, isononyl isononanoate, tridecyl neopentanoate, isocetyl neopentanoate, isostearyl neopentanoate, octyldodecyl neopentanoate and isoarachidyl neopentanoate.

Preferably, the one or more liquid fatty esters are selected from isopropyl myristate, methyl myristate, ethyl myristate, butyl myristate, isobutyl myristate, 2-octyldodecyl myristate, 2-ethylhexyl monococoate (or octyl monococoate), ethyl palmitate, isopropyl palmitate, isobutyl palmitate, 2-ethylhexyl palmitate (or octyl palmitate), butyl stearate, isopropyl stearate, isobutyl stearate, isocetyl stearate, isostearyl isostearate, isopropyl isostearate, 2-ethylhexyl stearate (or octyl stearate), isononyl isononanoate, 2-ethylhexyl hydroxystearate (or octyl hydroxystearate) and decyl oleate. More preferably, the one or more liquid fatty esters are selected from butyl stearate, isopropyl stearate, isopropyl isostearate, isobutyl stearate, isocetyl stearate, isostearyl isostearate, isopropyl isostearate, 2-ethylhexyl stearate (or octyl stearate), 2-ethylhexyl hydroxystearate (or octyl hydroxystearate), decyl oleate, isononyl isononanoate, isopropyl myristate, isononyl isononanoate, and isopropyl palmitate.

The total amount of the one or more fatty esters, if present, will vary but is typically about 1 to about 20 wt. %, based on the total weight of the anhydrous hair treatment composition. In further embodiments, the anhydrous hair treatment composition includes about 1 to about 18 wt. %, about 1 to about 15 wt. %, about 1 to about 12 wt. %, about 2 to about 20 wt. %, about 2 to about 18 wt. %, about 2 to about 15 wt. %, about 2 to about 12 wt. %, about 5 to about 20 wt. %, about 5 to about 18 wt. %, about 5 to about 15 wt. %, about 5 to about 12 wt. %, about 8 to about 20 wt. %, about 8 to about 18 wt. %, about 8 to about 15 wt. %, or about 8 to about 12 wt. % of the one or more fatty esters, based on the total weight of the anhydrous hair treatment composition. Preferably, the anhydrous hair treatment composition includes about 2 to about 18 wt. %, more preferably about 4 to about 16 wt. %, and even more preferably about 6 to about 14 wt. % of the one or more fatty esters, based on the total weight of the anhydrous hair treatment composition.

(e)(iii) Fatty Carbonates

The anhydrous hair treatment composition may optionally include one or more fatty carbonates. Fatty carbonates are also known as “fatty carbonate esters.” For purposes of the instant disclosure, fatty carbonates (or fatty carbonate esters) are separate elements from the “fatty esters” discussed above. A fatty carbonate is a carbonate moiety accompanied by one or two alkyl groups having at least 6 carbon atoms. Fatty carbonates may be represented by the formula R₈O(C═O)OR₉, wherein R₈ and Ry are independently linear or branched, saturated or unsaturated alkyl chains, wherein at least one of R₈ and R₉ is an alkyl group having at least 6 carbon atoms. For example, R₈ and R₉ independently have 1 to 30 carbon atoms, preferably 2 to 28 carbon atoms, more preferably 4 to 24 carbon atoms, or even more preferably 6 to 22 carbon atoms, provided that at least one of R₈ and R₉ is an alkyl group having at least 6 carbon atoms. Preferably, the one or more fatty carbonates are selected from C14-15 dialkyl carbonate, dicaprylyl carbonate, dipentyl carbonate, dimyristyl carbonate diethylhexyl carbonate, dipropylheptyl carbonate, didodecyl carbonate, ethyl dodecyl carbonate, diheptyl carbonate, didecyl carbonate, and didodecyl carbonate.

In a preferred embodiment, the anhydrous hair treatment composition includes dicaprylyl carbonate.

The total amount of the one or more fatty carbonates, if present, in the anhydrous hair treatment composition, if present, will vary but is typically about 1 to about 20 wt. %, based on the total weight of the anhydrous hair treatment composition. In further embodiments, the anhydrous hair treatment composition includes about 1 to about 15 wt. %, about 1 to about 12 wt. %, about 1 to about 10 wt. %, about 1 to about 8 wt. %, about 2 to about 20 wt. %, about 2 to about 15 wt. %, about 2 to about 12 wt. %, about 2 to about 10 wt. %, about 2 to about 8 wt. %, about 4 to about 20 wt. %, about 4 to about 15 wt. %, about 4 to about 12 wt. %, about 4 to about 10 wt. %, about 4 to about 8 wt. %, about 5 to about 20 wt. %, about 5 to about 15 wt. %, about 5 to about 10 wt. %, or about 5 to about 10 wt. % of the one or more fatty carbonates, based on the total weight of the anhydrous hair treatment composition. Preferably, the anhydrous hair treatment composition includes about 1 to about 18 wt. %, more preferably about 2 to about 15 wt. %, and even more preferably about 4 to about 10 wt. % of one or more fatty carbonates, based on the total weight of the anhydrous hair treatment composition.

(e)(iv) Caprylic/Capric Triglyceride

The anhydrous hair treatment compositions may optionally include caprylic/capric triglyceride. Caprylic/capric triglyceride is a clear, odorless, liquid that derived from coconut oil and glycerin. The total amount of the caprylic/capric triglyceride in the anhydrous hair treatment composition, if present, will vary but is typically about 1 to about 25 wt. %, based on the total weight of the anhydrous hair treatment composition. In further embodiments, the anhydrous hair treatment composition includes from about 1 to about 20 wt. %, about 1 to about 18 wt. %, about 1 to about 15 wt. %, about 1 to about 12 wt. %, about 1 to about 10 wt. %, about 1 to about 8 wt. %, about 1 to about 5 wt. %, about 5 to about 20 wt. %, about 5 to about 18 wt. %, about 5 to about 15 wt. %, about 5 to about 12 wt. %, about 8 to about 25 wt. %, about 8 to about 20 wt. %, about 8 to about 18 wt. %, about 8 to about 15 wt. %, or about 8 to about 12 wt. %, of the caprylic/capric triglycerides, based on the total weight of the anhydrous hair treatment composition. In a preferred embodiment, the anhydrous hair treatment composition includes about 1 to about 25 wt. %, preferably about 5 to about 25 wt. %, and more preferably about 8 to about 12 wt. %, based on the total weight of the anhydrous hair treatment composition.

In a preferred embodiment, the anhydrous hair treatment composition includes a plurality of emollients, wherein the plurality of emollients comprises, consists essentially of, or consists of one or more fatty ethers, one or more fatty esters, one or more fatty carbonates, and/or caprylic/capric triglyceride. For example, the plurality of emollients may comprise, consist essentially of, or consist of:
(e)(i) optionally, about 1 to about 18 wt. %, more preferably about 2 to about 16 wt. %, and even more preferably about 6 to about 14 wt. % of the one or more fatty ethers, preferably one or more dialkyl fatty ethers, more preferably, wherein the one or more dialkyl fatty ethers are selected from dicaprylyl ether, didecyl ether, dimyristyl ether, distearyl ether, dilauryl ether, dihexyl ether, dioctyl ether, diethylhexyl ether, or dioctyl ether, dodecyl ether, wherein dicaprylyl ether is particularly preferred; and/or
(e)(ii) optionally, about 1 to about 18 wt. %, more preferably about 2 to about 16 wt. %, and even more preferably about 6 to about 14 wt. % of the one or more fatty esters, preferably selected from ethyl laurate, butyl laurate, hexyl laurate, isohexyl laurate, isopropyl laurate, methyl myristate, ethyl myristate, butyl myristate, isobutyl myristate, isopropyl myristate, 2-octyldodecyl myristate, 2-ethylhexyl monococoate (or octyl monococoate), ethyl palmitate, isopropyl palmitate, isobutyl palmitate, 2-ethylhexyl palmitate (or octyl palmitate), butyl stearate, isopropyl stearate, isopropyl isostearate, isobutyl stearate, isocetyl stearate, isostearyl isostearate, isopropyl isostearate, 2-ethylhexyl stearate (or octyl stearate), 2-ethylhexyl hydroxystearate (or octyl hydroxystearate), decyl oleate, isononyl isononanoate, tridecyl neopentanoate, isocetyl neopentanoate, isostearyl neopentanoate, octyldodecyl neopentanoate and isoarachidyl neopentanoate, wherein isopropyl isostearate is particularly preferred; and/or
(e)(iii) optionally, about 1 to about 18 wt. %, more preferably about 2 to about 15 wt. %, and even more preferably about 4 to about 10 wt. % of one or more fatty carbonates, preferably one or more fatty carbonates selected from C14-15 dialkyl carbonate, dicaprylyl carbonate, dipentyl carbonate, dimyristyl carbonate diethylhexyl carbonate, dipropylheptyl carbonate, didodecyl carbonate, ethyl dodecyl carbonate, diheptyl carbonate, didecyl carbonate, or didodecyl carbonate, wherein dicaprylyl carbonate is particularly preferred.
(e)(iv) optionally, about 1 to about 20 wt. %, preferably about 5 to about 18 wt. %, and more preferably about 8 to about 15 wt. % of caprylic/capric triglyceride.

### (f) Miscellaneous Ingredients

The anhydrous hair treatment composition optionally includes one or more miscellaneous ingredients. Miscellaneous ingredients are ingredients that are compatible with the anhydrous hair treatment composition and do not disrupt or materially affect the basic and novel properties of the anhydrous hair treatment composition. Miscellaneous ingredients commonly used in cosmetics and hair care products are known in the art. Nonlimiting examples include preservatives, fragrances, pH adjusters, salts, chelating agents, buffers, antioxidants, flavonoids, vitamins, botanical extracts, UV filtering agents, proteins, protein hydrolysates, emollients, humectants, hydrotropes, fillers (e.g., organic or inorganic, such as talk, calcium carbonate, dextrin, silica, etc.), pearlescent agents, composition colorants, etc.

In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair.

In addition to the nonlimiting list of miscellaneous ingredients set forth above, components described as optional throughout the instant disclosure may be characterized as miscellaneous ingredients if not expressly set forth as a required component of the anhydrous hair treatment composition. Thus, the term “miscellaneous ingredient” is understood as a catch-all phrase representing additional components that may optionally be present in amounts designated for the miscellaneous ingredients.

The total amount of miscellaneous ingredients in the anhydrous hair treatment composition, if present, will vary. Nonetheless, the anhydrous hair treatment composition typically includes from about 0.01 to about 10 wt. % of one or more miscellaneous ingredients, based on the total weight of the anhydrous hair treatment composition. In further embodiments, the anhydrous hair treatment composition includes about 0.01 to about 8 wt. %, about 0.01 to about 5 wt. %, about 0.01 to about 3 wt. %, about 0.1 to about 10 wt. %, about 0.1 to about 8 wt. %, about 0.1 to about 5 wt. %, about 0.1 to about 3 wt. %, about 1 to about 10 wt. %, about 1 to about 8 wt. %, about 1 to about 5 wt. %, or about 1 to about 3 wt. % of one or more miscellaneous ingredients, based on the total weight of the anhydrous hair treatment composition.

### Polymeric Oil Thickening Agents

The anhydrous hair treatment composition optionally includes one or more polymeric oil thickening agents. Likewise, in various embodiments, the anhydrous hair treatment composition is free or essentially free from polymeric oil thickening agents. The term “polymeric oil thickening agent” means a polymer which when combined with other components of the anhydrous hair treatment composition results in a thickening of the anhydrous hair treatment composition. Nonlimiting examples of oil thickening agents include polymers and copolymers, gums, and polyethylenes. Non-polymeric oil thickening agents can optionally be included in the anhydrous hair treatment compositions, but they are not required. Nonlimiting examples of non-polymeric oil thickening agents include organoclays, silica, silica dimethyl silylate. In various embodiments, the anhydrous hair treatment composition is preferably free or essentially free from non-polymeric oil thickening agents. In other embodiments, in addition to the one or more polymeric oil thickening agents, the anhydrous hair treatment composition preferably includes one or more non-polymeric oil thickening agents.

Polymeric oil thickening agents include, for example, a hydrophobic polymer that is a reaction product of a natural or food-derived oil and an acrylate or methacrylate polymer, preferably a methacrylate polymer. The natural or food-derived oil may be a drying oil or a semi-drying oil. Nonlimiting examples include linseed oil, sunflower oil, tung oil, fish oil, cottonseed oil, and soybean oil. The methacrylate polymer can be formed from methacrylate monomers, for example, monomers selected from isobutyl methacrylate, methyl methacrylate, ethyl methacrylate, and n-butyl methacrylate. In a preferred embodiment, the hydrophobic polymer is formed from a natural or food-derived oil and an isobutyl methacrylate polymer. For example, the hydrophobic polymer may be the reaction product of about 72 to about 77 parts by weight of the natural or food-derived oil and about 23 to about 28 parts by weight of a methacrylate polymer. More specifically, the hydrophobic polymer may be the reaction product of linseed oil and poly(isobutyl methacrylate) in a suitable solvent, such as, for example, 2,2,4-trimethyl-1,3-pentanediol monoisobutyrate. Preferably, the reaction product is formed from about 72 to about 77% of linseed oil and about 23 to about 28% of isobutyl methacrylate polymer in a suitable solvent, such as 2,2,4-trimethyl-1,3-pentanediol monoisobutyrate, which is poly(linseed oil/isobutyl methacrylate).

The one or more polymeric oil thickening agents may include amorphous polymers formed by polymerization of an olefin. The olefin can be an ethylenically unsaturated elastomer monomer. Examples of olefins include, but are not limited to, ethylenic carbide monomers, for example those comprising one or two ethylenic unsaturations, comprising from 2 to 5 carbon atoms, such as ethylene, propylene, butadiene, or isoprene.

The one or more polymeric oil thickening agents may include polycondensation products of the polyamide type resulting from condensation between (α) at least one acid chosen from dicarboxylic acids comprising at least 32 carbon atoms, such as the dimeric fatty acids and (β) alkylenediamines, such as ethylenediamine, wherein the polymeric polyamide comprises at least one terminal carboxylic acid group esterified or amidated with at least one monoalcohol or monoamine comprising from 12 to 30 linear and saturated carbon atoms, for example ethylenediamine/stearyl dilinoleate copolymers such as that marketed under the name Uniclear 100 VG® by Arizona Chemical.

Nonlimiting examples of preferred polymeric oil thickening agents include poly C10-30 alkyl acrylate, VP/hexadecene copolymer, trimethylpentanediol/adipic acid/glycerin crosspolymer, ethylene/propylene/styrene copolymer, butylene/ethylene/styrene copolymer, VP/eicosene copolymer, bis-dioctadecylamide dimer dilinoleic acid/ethylenediamine copolymer, cetearyl dimethicone crosspolymer, hydrogenated poly(C6-20 olefin), HDI/trimethylol hexyllactone crosspolymer, dimethicone/vinyl dimethicone crosspolymer, and vinyl dimethicone/methicone silsesquioxane crosspolymer. In various embodiments, preferred polymeric oil thickening agents include hydrogenated poly(C6-20 olefin), HDI/trimethylol hexyllactone crosspolymer, and poly(linseed oil/isobutyl methacrylate). A combination of hydrogenated poly(C6-20 olefin) and HDI/trimethylol hexyllactone crosspolymer is a particularly preferred polymeric oil thickening agent.

The total amount of the one or more polymeric oil thickening agents in the anhydrous hair treatment composition, if present, will vary but is typically about 0.05 to about 5 wt. %, based on the total weight of the anhydrous hair treatment composition. In further embodiments, the anhydrous hair treatment composition includes about 0.05 to about 4 wt. %, about 0.05 to about 3 wt. %, about 0.05 to about 2 wt. %, about 0.05 to about 1 wt. %, about 0.1 to about 5 wt. %, about 0.1 to about 4 wt. %, about 0.1 to about 3 wt. %, about 0.1 to about 2 wt. %, about 0.1 to about 1 wt. %, about 0.2 to about 5 wt. %, about 0.2 to about 4 wt. %, about 0.2 to about 3 wt. %, about 0.2 to about 1 wt. % of the one or more polymeric oil thickening agents, based on the total weight of the anhydrous hair treatment composition. Preferably, the anhydrous hair treatment composition includes about 0.05 to about 5 wt. %, more preferably about 0.05 to about 3 wt. %, and even more preferably about 0.1 to about 2 wt. % of the one or more polymeric oil thickening agents, based on the total weight of the anhydrous hair treatment composition.

### Methods for Treating Hair

As its name suggests, the anhydrous hair treatment composition is particularly useful in methods for treating hair, especially human hair of the head. The anhydrous hair treatment composition is useful as both a rinse-off product and a leave-on products. A “rinse-off” product refers to a composition that is rinsed and/or washed from the hair with water either after or during the application of the composition onto the hair, and before drying and/or styling the hair. At least a portion of the composition is removed from the hair during the rinsing and/or washing. A “leave-on” product refers to a composition that is not rinsed and/or washed from the hair after or during application of the composition onto the hair. The composition remains on the hair during drying and/or throughout styling.

Methods of using the anhydrous hair treatment composition include methods for caring for hair, methods for conditioning hair, methods for hydrating or moisturizing hair, methods for smoothing hair, methods for reducing frizz of hair, and methods for enhancing shine of hair. The methods include applying an effective amount of the anhydrous hair treatment composition to the hair. For example, the anhydrous hair treatment composition may be applied to hair in need of conditioning, hair in need of hydration or moisturization, hair in need of smoothing, hair susceptible to frizz or hair suffering from frizz, hair in need of shine, or a combination thereof. Moreover, a preferred embodiment, the anhydrous hair treatment composition is applied to damaged hair, for example, damaged hair in need of strengthening, in need of hydrating or moisturizing, in need of conditioning, in need of smoothing, and/or in need of frizz control.

In a preferred embodiment, the anhydrous hair treatment composition is applied to wet or damp hair, for example, freshly washed and optionally conditioned hair. After rinsing a shampoo and optionally rinsing a conditioner from the hair, while the hair remains wet or damp, an effective amount of the anhydrous hair treatment composition is applied to the wet or damp hair. Upon application, the anhydrous hair treatment composition can be massaged, combed, or otherwise spread throughout the hair and the scalp. Preferably, the anhydrous hair treatment composition is spread throughout the totality of the hair, including to the ends of the hair. After the anhydrous hair treatment composition has been spread throughout the hair, it can be rinsed from the hair or the hair can be cleansed with a shampoo and optionally treated with a conditioner. In this embodiment, the anhydrous hair treatment composition is a rinse-out product. Before rinsing the anhydrous hair treatment composition from the hair or shampooing the hair, the anhydrous hair treatment composition is preferably allowed to remain on the hair for a period of time, for example, for about 30 second to about 30 minutes. The period of time may also be from about 1 minute to about 30 minutes, about 1 minute to about 15 minutes, about 1 minute to about 10 minutes, about 1 minute to about 5 minutes, or about 5 minutes to about 15 minutes.

Preferably, the anhydrous hair treatment composition is not rinsed or cleansed from the hair but is applied as a leave-on product. After spreading the anhydrous hair treatment composition throughout the wet or damp hair, the anhydrous hair treatment composition is not rinsed or cleansed from the hair. Instead, the hair upon which the anhydrous hair treatment composition is applied, is allowed to dry or is forcibly dried, for example, using a blow drying and the hair is styled. Alternatively, the hair may be styled while still wet or damp and subsequently dried.

### Preferred Embodiments

In a preferred embodiment, the anhydrous hair treatment composition comprises, consists essentially of, or consist of:
(a) about 10 to about 95 wt. %, preferably about 40 to about 65 wt. %, more preferably about 50 to about 65 wt. % of a plurality of vegetable oils, wherein the plurality of vegetable oils comprises:
   at least 10 wt. %, preferably at least 25 wt. %, more, preferably about 25 to about 55 wt. %, more preferably about 30 to about 50 wt. % of castor oil, and even more preferably about 35 to about 45 wt. % of castor oil,
   coconut oil, preferably wherein the coconut oil is in an amount of about 1 to about 40 wt. %, more preferably about 5 to about 30 wt. %, and even more preferably about 5 to about 15 wt. %, and
   optionally, one or more further vegetable oils (other than the castor oil and the coconut oil), preferably in an amount of about 1 to about 30 wt. %, more preferably about 2 to about 20 wt. %, and even more preferably about 5 to about 15 wt. %;
(b) about 0.1 to about 10 wt. %, preferably about 0.5 to about 6 wt. %, more preferably about 1 to about 4 wt. % of one or more cationic surfactants, wherein the one or more cationic surfactants are preferably selected from quaternary ammonium surfactants and amidoamine surfactants, wherein the one or more cationic surfactants are preferably selected from one or more amidoamine surfactants, wherein the one or more amidoamine surfactants are preferably selected from oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethylamine, behenamidoethyldimethylamine, arachidamidopropyldimethylamine, arachidamido-propyidiethylamine, and arachidamidoethyidiethylamine, arachidamidoethyidimethylamine, more preferably wherein the one or more amidoamine surfactants comprises or consist of stearamidopropyl dimethylamine;
(c) about 0.1 to about 12 wt. %, preferably about 0.5 to about 10 wt. %, more preferably about 1 to about 8 wt. %, and even more preferably about 3 to about 8 wt. % of one or more fatty alcohols, wherein preferably the one or more fatty alcohols are selected from fatty alcohols having from 12 to 34 carbon atoms and are branched or linear and saturated or unsaturated, wherein more preferably, the anhydrous hair treatment composition includes a plurality of fatty alcohols, wherein preferably, the plurality of fatty alcohols include one or more solid fatty alcohol and one or more liquid fatty alcohols, wherein
   the one or more solid fatty alcohols are selected from stearyl alcohol, cetyl alcohol, cetearyl alcohol alcohol, myristyl alcohol, behenyl alcohol, and arachidyl alcohol;
   the one or more liquid fatty alcohols selected from isostearyl alcohol, oleyl alcohol, octyldodecanol, linoleyl alcohol, linolenyl alcohol, lauryl alcohol, 2-hexyldecanol, 2-heptyldecanol, enanthic alcohol, caprylic alcohol, pelargonic alcohol, and capric alcohol;
(d) optionally, about 0.01 to about 5 wt. %, preferably about 0.05 to about 4 wt. %, more preferably about 0.1 to about 4 wt. % of one or more sensorially active agents, wherein the one or more sensorially active agents are preferably selected from essential oils, cooling agents, warming agents, terpenes, and terpinoids;
(e) optionally, about 1 to about 55 wt. %, preferably about 15 to about 50 wt. %, more preferably about 20 to about 50 wt. %, even more preferably about 25 to about 45 wt. % of one or more emollients, wherein the one or more emollients are preferably selected from fatty ethers, fatty esters, fatty carbonates, propylene glycol esters of fatty acids, and caprylic/capric triglyceride; and
(f) optionally, about 0.01 to about 10 wt. %, preferably about 0.1 to about 8 wt. %, more preferably about 1 to about 5 wt. % of one or more miscellaneous ingredients, wherein the one or more miscellaneous ingredients are preferably selected from preservatives, fragrances, chelating agents, antioxidants, flavonoids, vitamins, botanical extracts, UV filtering agents, proteins, protein hydrolysates, protein isolates, fillers, and composition colorants;
   wherein all weight percentages are based on a total weight of the composition.

The anhydrous hair treatment composition is preferably a homogenous, transparent solution. In addition, the anhydrous hair treatment composition preferably forms a lamellar structure when contacted with water.

In a further preferred embodiment, the anhydrous hair treatment composition comprises, consists essentially of, or consist of:
(a) about 10 to about 95 wt. %, preferably about 40 to about 65 wt. %, more preferably about 50 to about 65 wt. % of a plurality of vegetable oils, wherein the plurality of vegetable oils comprises:
   at least 10 wt. %, preferably at least 25 wt. %, more, preferably about 25 to about 55 wt. %, more preferably about 30 to about 50 wt. % of castor oil, and even more preferably about 35 to about 45 wt. % of castor oil,
   coconut oil, preferably wherein the coconut oil is in an amount of about 1 to about 40 wt. %, more preferably about 5 to about 30 wt. %, and even more preferably about 5 to about 15 wt. %, and
   optionally, one or more further vegetable oils (other than the castor oil and the coconut oil), preferably in an amount of about 1 to about 30 wt. %, more preferably about 2 to about 20 wt. %, and even more preferably about 5 to about 15 wt. %, preferably wherein the further vegetable oil is soybean oil;
(b) about 0.1 to about 10 wt. %, preferably about 0.5 to about 6 wt. %, more preferably about 1 to about 4 wt. % of one or more cationic surfactants, wherein the one or more cationic surfactants are preferably selected from quaternary ammonium surfactants and amidoamine surfactants, wherein the one or more cationic surfactants are preferably selected from one or more amidoamine surfactants, wherein the one or more amidoamine surfactants are preferably selected from oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethylamine, behenamidoethyldimethylamine, arachidamidopropyldimethylamine, arachidamido-propyidiethylamine, and arachidamidoethyidiethylamine, arachidamidoethyidimethylamine, more preferably wherein the one or more amidoamine surfactants comprises or consist of stearamidopropyl dimethylamine;
(c) about 0.1 to about 12 wt. %, preferably about 0.5 to about 10 wt. %, more preferably about 1 to about 8 wt. %, and even more preferably about 3 to about 8 wt. % of one or more fatty alcohols, wherein preferably the one or more fatty alcohols are selected from fatty alcohols having from 12 to 34 carbon atoms and are branched or linear and saturated or unsaturated, wherein more preferably, the anhydrous hair treatment composition includes a plurality of fatty alcohols, wherein preferably, the plurality of fatty alcohols include one or more solid fatty alcohol and one or more liquid fatty alcohols, wherein
   the one or more solid fatty alcohols are selected from stearyl alcohol, cetyl alcohol, cetearyl alcohol alcohol, myristyl alcohol, behenyl alcohol, and arachidyl alcohol;
   the one or more liquid fatty alcohols selected from isostearyl alcohol, oleyl alcohol, octyldodecanol, linoleyl alcohol, linolenyl alcohol, lauryl alcohol, 2-hexyldecanol, 2-heptyldecanol, enanthic alcohol, caprylic alcohol, pelargonic alcohol, and capric alcohol;
(d) optionally, about 0.01 to about 5 wt. %, preferably about 0.05 to about 4 wt. %, more preferably about 0.1 to about 4 wt. % of one or more sensorially active agents, wherein the one or more sensorially active agents are preferably selected from essential oils, cooling agents, warming agents, terpenes, and terpinoids;
(e) about 20 to about 50 wt. %, preferably about 25 to about 45 wt. %, more preferably about 30 to about 45 wt. % of a plurality of emollients, wherein the plurality of emollients comprises:
   Optionally, about 2 to about 18 wt. %, more preferably about 4 to about 16 wt. %, and even more preferably about 6 to about 14 wt. % of one or more fatty ethers, wherein the one or more fatty ethers are preferably selected from dialkyl fatty ethers, more preferably, wherein the one or more dialkyl fatty ethers are selected from dicaprylyl ether, didecyl ether, dimyristyl ether, distearyl ether, dilauryl ether, dihexyl ether, dioctyl ether, diethylhexyl ether, dioctyl ether, and dodecyl ether, wherein dicaprylyl ether is particularly preferred;
   optionally, about 2 to about 18 wt. %, more preferably about 4 to about 16 wt. %, and even more preferably about 6 to about 14 wt. % of the one or more fatty esters, wherein the one or more fatty esters is preferably selected from ethyl laurate, butyl laurate, hexyl laurate, isohexyl laurate, isopropyl laurate, methyl myristate, ethyl myristate, butyl myristate, isobutyl myristate, isopropyl myristate, 2-octyldodecyl myristate, 2-ethylhexyl monococoate (or octyl monococoate), ethyl palmitate, isopropyl palmitate, isobutyl palmitate, 2-ethylhexyl palmitate (or octyl palmitate), butyl stearate, isopropyl stearate, isopropyl isostearate, isobutyl stearate, isocetyl stearate, isostearyl isostearate, isopropyl isostearate, 2-ethylhexyl stearate (or octyl stearate), 2-ethylhexyl hydroxystearate (or octyl hydroxystearate), decyl oleate, isononyl isononanoate, tridecyl neopentanoate, isocetyl neopentanoate, isostearyl neopentanoate, octyldodecyl neopentanoate and isoarachidyl neopentanoate, wherein isopropyl isostearate is particularly preferred;
   optionally, about 1 to about 18 wt. %, more preferably about 2 to about 15 wt. %, and even more preferably about 4 to about 10 wt. % of one or more fatty carbonates, preferably wherein the one or more fatty carbonates are selected from C14-15 dialkyl carbonate, dicaprylyl carbonate, dipentyl carbonate, dimyristyl carbonate diethylhexyl carbonate, dipropylheptyl carbonate, didodecyl carbonate, ethyl dodecyl carbonate, diheptyl carbonate, didecyl carbonate, or didodecyl carbonate, wherein dicaprylyl carbonate is particularly preferred; and
   optionally, about 1 to about 20 wt. %, preferably about 5 to about 20 wt. %, more preferably about 6 to about 15 wt. %, and even more preferably about 8 to about 12 wt. % of caprylic/capric triglyceride;
(f) optionally, about 0.01 to about 10 wt. %, preferably about 0.1 to about 8 wt. %, more preferably about 1 to about 5 wt. % of one or more miscellaneous ingredients, wherein the one or more miscellaneous ingredients are preferably selected from preservatives, fragrances, chelating agents, antioxidants, flavonoids, vitamins, botanical extracts, UV filtering agents, proteins, protein hydrolysates, protein isolates, fillers, or composition colorants;
   wherein all weight percentages are based on a total weight of the composition.

The anhydrous hair treatment composition is preferably a homogenous, transparent solution. In addition, the anhydrous hair treatment composition preferably forms a lamellar structure when contacted with water.

In a further preferred embodiment, the anhydrous hair treatment composition comprises, consists essentially of, or consist of:
(a) about 10 to about 90 wt. %, preferably about 35 to about 70 wt. %, more preferably about 40 to about 65 wt. %, and more preferably about 50 to about 65 wt. % of a plurality of vegetable oils, wherein the plurality of vegetable oils comprises:
   at least 10 wt. %, preferably about 15 to about 90 wt. %, more preferably about 25 to about 55 wt. %, even more preferably about 30 to about 50 wt. %, and even more preferably about 35 to about 45 wt. % of castor oil,
   about 1 to about 40 wt. %, more preferably about 5 to about 30 wt. %, and even more preferably about 5 to about 15 wt. % of coconut oil, and
   optionally, one or more further vegetable oils (other than the castor oil and the coconut oil), preferably in an amount of about 1 to about 30 wt. %, more preferably about 2 to about 20 wt. %, and even more preferably about 5 to about 15 wt. %, wherein one of the one or more further vegetable oils is preferably selected from arachis oil, amaranth seed oil, apricot oil, argan oil, avocado oil, babassu oil, cottonseed oil, borage seed oil, camelina oil, camellia oil, safflower oil, peanut oil, pomegranate seed oil, grapefruit seed oil, hemp oil, hazelnut oil, elderberry seed oil, currant seed oil, jojoba oil, linseed oil, macadamia nut oil, corn oil, almond oil, manila oil, evening primrose oil, olive oil, palm oil, palm kernel oil, brazil nut oil, pecan nut oil, peach kernel oil, rapeseed oil, rosehip oil, sea buckthorn fruit oil, sea buckthorn kernel oil, sesame oil, soybean oil, sunflower oil, grapeseed oil, walnut oil, wild rose oil, or wheat germ oil, wherein soybean oil is particularly preferred;
(b) about 0.1 to about 10 wt. %, preferably about 0.5 to about 6 wt. %, more preferably about 1 to about 4 wt. % of one or more cationic surfactants, wherein the one or more cationic surfactants are preferably selected from quaternary ammonium surfactants and amidoamine surfactants, wherein the one or more cationic surfactants are preferably selected from one or more amidoamine surfactants, wherein the one or more amidoamine surfactants are preferably selected from oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethylamine, behenamidoethyldimethylamine, arachidamidopropyldimethylamine, arachidamido-propyidiethylamine, arachidamidoethyidiethylamine, or arachidamidoethyidimethylamine, more preferably wherein the one or more amidoamine surfactants comprises or consist of stearamidopropyl dimethylamine;
(c) about 0.1 to about 12 wt. %, preferably about 0.5 to about 10 wt. %, more preferably about 1 to about 8 wt. %, and even more preferably about 3 to about 8 wt. % of one or more fatty alcohols, wherein preferably the one or more fatty alcohols are selected from fatty alcohols having from 12 to 34 carbon atoms and are branched or linear and saturated or unsaturated, wherein more preferably, the anhydrous hair treatment composition includes a plurality of fatty alcohols, wherein preferably, the plurality of fatty alcohols include one or more solid fatty alcohol and one or more liquid fatty alcohols, wherein
   the one or more solid fatty alcohols are selected from stearyl alcohol, cetyl alcohol, cetearyl alcohol alcohol, myristyl alcohol, behenyl alcohol, and arachidyl alcohol;
   the one or more liquid fatty alcohols selected from isostearyl alcohol, oleyl alcohol, octyldodecanol, linoleyl alcohol, linolenyl alcohol, lauryl alcohol, 2-hexyldecanol, 2-heptyldecanol, enanthic alcohol, caprylic alcohol, pelargonic alcohol, and capric alcohol;
(d) optionally, about 0.01 to about 5 wt. %, preferably about 0.05 to about 4 wt. %, more preferably about 0.1 to about 4 wt. % of one or more sensorially active agents, wherein the one or more sensorially active agents are preferably selected from essential oils, cooling agents, warming agents, terpenes, or terpinoids,
   wherein the one or more essential oils are selected from lavender oil, geranium oil, juniper berry oil, bay leaf oil, lemon oil, mandarin orange oil, orange oil, absinthe oil, African bluegrass oil, ajowan oil, anethi oil, angelica root oil, anise star oil, arborvitae wild oil, armoise mugwort oil, asafetida oil, Australian balm oil, basil oil, benzoin oil, bergamot calabrian oil, betel leaf oil, birch sweet oil, birch tar oil, black currant-seed oil, blood orange oil, buchu oil, cabreuva oil, cade oil, cajeput oil, calamus root oil, camphor oil, cananga oil, caraway oil, cardamom oil, carrotseed oil, cassia oil, cedarleaf oil cedarwood atles oil, cedarwood Chinese oil, cedarwood Himalayan oil, cedarwood texas oil, cedarwood Virginian oil, celery seed oil, chamomile German oil, chamomile moroc oil, chamomile roman oil, chaulmogra oil, chenopodium oil, chilly seed oil, cinnamon bark oil, cinnamon leaf oil, citral oil, citronelia Ceylon oil, clarysage oil, clementine oil, clove bud oil, clove leaf oil, cognac oil, copaiba balsam oil, coriander oil, costus root oil, coffee oil, cumin oil, curcuma oil, curry leaf oil, cypress Australian blue oil, cypress French cypriol oil, davana oil, dill seed oil, dill weed oil, elemi oil, emu oil, eucalyptus oil, fennel bitter oil, fenugreek oil, fir balsam oil, fir needle oil, flax seed organic oil, frankincense oil garlic oil, galbanum oil, ginger oil, ginger grass oil, gaia oil, grapefruit pink oil, grapefruit white oil, guaiacwood oil, helichrysum oil, horseradish oil, hydacheim oil, hyssop oil, jamarosa root oil, Himalayan wild oil, karanj seed oil, kanuka oil, katrafay oil, key lime oil, kunzea oil, kukuinut oil, lantana oil, laurel leaf oil, lemongrass oil, lemon eucalyptus oil, lemon myrtle oil, lemon 5 fold oil, lican oil, lime oil, Litsea cubeba oil, lovage leaf oil, mace oil, mandarin oil, manuka oil, marjoram oil, Melissa leaf oil, menthol liquid oil, mullein oil, myrrh oil, myrtle oil, neem oil, neroli oil, nerolina Australian oil, niaouli oil, noni oil, nutmeg oil, oat oil, onion oil, opoponax oil, orange bitter oil, orange sweet oil, origanum oil, orange 5 fold oil, palmarosa oil, parsley oil, patchouli oil, pennyroyal oil, pepper oil, peppermint oil, peru balsam oil, petitgrain oil, pimento leaf oil, pine long leaf oil, pine scotch oil, pine commercial oil, pomegranate oil, pomouwood oil, ravensara wild oil, rosalina Australian oil, rose geranium oil, rosemary oil, rosewood oil, rue oil, sage Dalmatian oil, sandalwood oil, savine oil, savory winter oil, sea buckthorn oil, spearmint oil, spikenard oil, spruce oil, sugandh kokila oil, sumac oil, tagetes oil, tangerine oil, tansy blue oil, tarragon oil, tangerine 5 fold oil, tea tree oil, tea tree lemon oil, thyme oil, tolu balsam oil, turmeric oil, valerian root oil, vanilla 10 fold oil, vetiver oil, wintergreen oil, wild verbena oil, wormwood oil, yarrow oil, or ylang ylang oil, wherein more preferably, the one or more essential oils are selected from lavender oil, peppermint oil, rosemary oil, tea tree oil, Roman chamomile oil, bergamot oil, jasmine oil, Ylang-ylang oil, eucalyptus oil, geranium oil, patchouli oil, lemon oil, frankincense oil, lemongrass oil, cedarwood oil, or grapefruit oil, and
   wherein the one or more cooling agents are selected from menthol, N-ethyl-p-menthyl-3-carboxiamide, cubebol, isopulegol, 3-(L-menthoxy) propane-1,2-diol, cis/trans-p-menthane-3,8-diol, 3-(L-menthoxy)-2-methylpropane-1,2-diol, 2-[2-(p-menthan-3-yloxy) ethoxy]ethanol, menthoxyethanol, (1R,2R,4R)-1-(2-hydroxy-4-methylcyclohexyl) ethenone, (1R,2R,5R)—N-ethyl-5-methyl-2-(prop-1-en-2-yl)-cyclohexanecarboxamide, N-(3-hydroxy-4-methoxyphenyl)-2-isopropyl-5,5-dimethyl-cyclohexanecarboxamide, N-[4-(cyanomethyl)phenyl]-2-isopropyl-5,5-dimethyl-cyclohexanecarboxamide, N-(2-Hydroxy-2-phenylethyl)-2-isopropyl-5,5-dimethyl-cyclohexane-1-carboxamide, L-menthol lactate, (−)-menthyl ethylene glycol carbonate, (−)-menthone 1,2-glycerol ketal, D/L-menthone 1,2-glycerol ketal, (−)-menthyl 1-propylene glycol carbonate, (−)-menthyl 2-propylene glycol carbonate, D/L-menthyl 1-propylene glycol carbonate, D/L-menthyl 2-propylene glycol carbonate, (1R,2S,5R)—N-(4-methoxyphenyl)-5-methyl-2-(1-methylethyl)cyclohexanecarboxamide, menthyl ethylamido oxalate, (E)-3-benzo[1,3]dioxol-5-yl-N,N-diphenyl-2-propenamide, 3,4-methylenedioxycinnamic acid, N,N-diphenylamide, N,N-dimethyl (−)-menthyl succinimide, (1R,2S,5R)—N-(4-(carbamoylmethyl)phenyl)-menthylcarboxamide, (−)-menthyl pyrrolidone carboxylate, L-phenylephrine p-menthane carboxamide, (1R,2S,5R)—N-(4-(cyanomethyl)-phenyl) menthylcarboxamide, (1R,2S,5R)—N-(2-(pyridin-2-yl)ethyl) menthylcarboxamide, 6-isopropyl-3,9-dimethyl-1,4-dioxaspiro[4.5]decan-2-one, (1R,2R,4S)-dihydroumbellulol, N-ethyl-p-menthane-3-carboxamide, 2-isopropyl-N,2,3-trimethylbutyramide, ethyl 3-(p-menthane-3-carboxamido)acetate, N-ethyl-2,2-diisopropylbutanamide, N-(1,1-dimethyl-2-hydroxyethyl)-2,2-dimethylbutanamide, N-cyclopropyl-5-methyl-2-isopropyl-cyclohexanecarbonecarboxamide, (−)-menthyl acetoacetate, (−)-menthyl succinate, (−)-menthyl (S)-3-hydroxybutyrate, (−)-menthyl glutarate, 1-[2-hydroxyphenyl]-4-[2-nitrophenyl]-1,2,3,6-tetrahydropyrimidine-2-one, di-(−)-menthyl glutarate, or N-(2-hydroxyethyl)-2,3-dimethyl-2-isopropylbutanamide, preferably wherein the one or more cooling agents includes menthol;
(e) optionally, about 1 to about 55 wt. %, preferably about 20 to about 50 wt. %, more preferably about 25 to about 45 wt. %, and even more preferably about 30 to about 45 wt. % of a plurality of emollients, wherein the plurality of emollients comprises:
   about 1 to about 20 wt. %, preferably about 2 to about 18 wt. %, more preferably about 4 to about 16 wt. %, and even more preferably about 6 to about 14 wt. % of one or more fatty ethers, wherein the one or more fatty ethers are preferably selected from dialkyl fatty ethers, more preferably, wherein the one or more dialkyl fatty ethers are selected from dicaprylyl ether, didecyl ether, dimyristyl ether, distearyl ether, dilauryl ether, dihexyl ether, dioctyl ether, diethylhexyl ether, dioctyl ether, or dodecyl ether, wherein dicaprylyl ether is particularly preferred;
   about 1 to about 20 wt. %, preferably about 2 to about 18 wt. %, more preferably about 4 to about 16 wt. %, and even more preferably about 6 to about 14 wt. % of the one or more fatty esters, wherein the one or more fatty esters is preferably selected from ethyl laurate, butyl laurate, hexyl laurate, isohexyl laurate, isopropyl laurate, methyl myristate, ethyl myristate, butyl myristate, isobutyl myristate, isopropyl myristate, 2-octyldodecyl myristate, 2-ethylhexyl monococoate (or octyl monococoate), ethyl palmitate, isopropyl palmitate, isobutyl palmitate, 2-ethylhexyl palmitate (or octyl palmitate), butyl stearate, isopropyl stearate, isopropyl isostearate, isobutyl stearate, isocetyl stearate, isostearyl isostearate, isopropyl isostearate, 2-ethylhexyl stearate (or octyl stearate), 2-ethylhexyl hydroxystearate (or octyl hydroxystearate), decyl oleate, isononyl isononanoate, tridecyl neopentanoate, isocetyl neopentanoate, isostearyl neopentanoate, octyldodecyl neopentanoate and isoarachidyl neopentanoate, wherein isopropyl isostearate is particularly preferred; and
   about 1 to about 18 wt. %, more preferably about 2 to about 15 wt. %, and even more preferably about 4 to about 10 wt. % of one or more fatty carbonates, preferably wherein the one or more fatty carbonates are selected from C14-15 dialkyl carbonate, dicaprylyl carbonate, dipentyl carbonate, dimyristyl carbonate diethylhexyl carbonate, dipropylheptyl carbonate, didodecyl carbonate, ethyl dodecyl carbonate, diheptyl carbonate, didecyl carbonate, or didodecyl carbonate, wherein dicaprylyl carbonate is particularly preferred;
   optionally, about 1 to about 25 wt. %, preferably about 5 to about 20 wt. %, more preferably about 6 to about 15 wt. %, and even more preferably about 8 to about 12 wt. % of caprylic/capric triglyceride; and
(f) optionally, about 0.01 to about 10 wt. %, preferably about 0.1 to about 8 wt. %, more preferably about 1 to about 5 wt. % of one or more miscellaneous ingredients, wherein the one or more miscellaneous ingredients are preferably selected from preservatives, fragrances, chelating agents, antioxidants, flavonoids, vitamins, botanical extracts, UV filtering agents, proteins, protein hydrolysates, protein isolates, fillers, or composition colorants;
   wherein all weight percentages are based on a total weight of the composition.

The anhydrous hair treatment composition is preferably a homogenous, transparent solution. In addition, the anhydrous hair treatment composition preferably forms a lamellar structure when contacted with water.

### Preferable Exclusions

The anhydrous hair treatment composition does not require silicones, including amino-functionalized silicones. Therefore, in a preferred embodiment, the anhydrous hair treatment composition is free or essentially free from silicones including amino-functionalized silicone.

The anhydrous hair treatment composition does not require cationic polymers. Therefore, in a preferred embodiment, the anhydrous hair treatment composition is free or essentially free from cationic polymers. In other embodiments, however, it can be useful to include one or more cationic polymers.

The anhydrous hair treatment composition is preferably free or essentially free from anionic surfactants, nonionic surfactants (other than fatty alcohols), and/or amphoteric surfactants. In another embodiment, the anhydrous hair treatment composition is free or essentially free from anionic surfactants. In another embodiment, the anhydrous hair treatment composition is free or essentially free from nonionic surfactants (other than fatty alcohols). In another embodiment, the anhydrous hair treatment composition is free or essentially free from amphoteric surfactants.

### EXAMPLES

Various changes can be made in the above-compositions and methods without departing from the scope of the invention. Accordingly, it is intended that the entire disclosure in the above description and in the examples given below, is illustrative and not limiting.

### Example 1

### Inventive Compositions

| | | | | A | B |
|---|---|---|---|---|---|
| (a) | Vegetable Oil | | CASTOR SEED OIL | 33.8 | 32 |
| | | | SOYBEAN OIL | 11.7 | |
| | | | COCONUT OIL | 9 | 10 |
| | | | SUNFLOWER SEED OIL | | 20 |
| Total (a) | | | | 54.5 | 62 |
| (b) | Cationic Surfactant | | STEARAMIDOPROPYL | 1.2 | 1.2 |
| | | | DIMETHYLAMINE | | |
| Total (b) | | | | 1.2 | 1.2 |
| (c) | Fatty | Solid | CETEARYL ALCOHOL | 3 | 3 |
| | Alcohols | | MYRISTYL ALCOHOL | 2.1 | 1.8 |
| | | | CETYL ALCOHOL | 0.06 | |
| | | Liquid | OLEYL ALCOHOL | 1 | 1 |
| | | | ISOSTEARYL ALCOHOL | 1 | |
| | | | LAURYL ALCOHOL | 0.7 | |
| Ratio of Solid FA:Liquid FA | | | | 1.9:1 | 4.8:1 |
| Total (c) | | | | 7.8 | 5.8 |
| Ratio of (b):(c) | | | | 1:6.5 | 1:4.8 |
| (d) | Sensorially | Essential | ROSEMARY LEAF OIL, | 0.8 | |
| | Active Agents | Oils | LAVENDER OIL, AND | | |
| | | | PEPPERMINT OIL | | |
| | | Terpenoid | MENTHOL | 1.1 | |
| (e) | Emollients | Triglyceride | CAPRYLIC/CAPRIC | 9 | |
| | | | TRIGLYCERIDE | | |
| | | Fatty Ester | ISOPROPYL ISOSTEARATE | 9 | |
| | | Fatty | DICAPRYLYL CARBONATE | 6.7 | 15 |
| | | Carbonate | | | |
| | | Fatty Ether | DICAPRYLYL ETHER | 9 | 15 |

| | | | |
|---|---|---|---|
| (f) | Miscellaneous* | ≤5 | ≤5 |

* Preservatives (tocopherol), fragrances, composition colorants, UV filters, etc.

### Example 2

### Comparative Composition

Inventive Composition A of Example 1 was compared with a popular commercial benchmark product (Commercial Benchmark #1), known for its excellent conditioning, smoothing, and anti-frizz properties. The Commercial Benchmark #1, like Inventive Composition A, is an oil-based product. However, it lacks cationic surfactants and fatty alcohols. The ingredients for the Commercial Benchmark #1 product are shown below.
Glycine soja (Soybean) Oil, Ricinus communis (Castor) Seed Oil, Rosmarinus officinalis (Rosemary) Leaf Oil, Simmondsia chinensis (Jojoba) Seed Oil, Mentha piperita (Peppermint) Oil, Eucalyptus globolus (Eucalyptus) Leaf Oil, Menthol, Melalueca alternifolia (Tea Tree) Leaf Oil, Cocos nucifera (Coconut) Oil, Equisetum arvense (Horsetail) Extract, Aloe Barbadensis Extract, Lavandula angustifolia (Lavender) Oil, Triticum vulgare (Wheat) Germ Oil, Carthamus tinctorius (Safflower) Seed Oil, Oenothera biennis (Evening Primrose) Oil, Vitis vinifera (Grape) Seed Oil, Benzyl Nicotinate, Prunus amygdalus Dulcis (Sweet Almond) Oil, Oryza sativa (Rice) Bran Oil, Tocopheryl Acetate, Biotin, Arctium lappa (Burdock) Root Extract, Glycerin, Apium graveolens (Celery) Seed Extract, Retinyl Palmitate, Cholecalciferol (Vitamin D), Ascorbic Acid, Ocimum, Basilicum (Basil) Oil, Pogostemon cablin (Patchouli) Oil, Salvia officinalis (Sage) Oil, Silica, Urtica dioica (Nettle) Extract.

After shampooing the hair of a volunteer, excess water was squeezed from the hair and the hair was towel dried. While still damp, equal amounts of Inventive Composition A and the Commercial Benchmark #1 were applied to opposite sides of a volunteer's head of hair. Both compositions were spread evenly throughout the hair from roots to tip. An expert evaluator then evaluated the attributes of the treated hair. Without rinsing the composition from the hair, the hair was dried with a blow dryer. After the hair was completely dried, the same expert evaluator again evaluated the attributes of the treated hair. The results are reported in the table below.

| | Inventive A | Benchmark #1 |
|---|---|---|
| Wet | Noticeably easier to | More volume |
| Attributes | distribute | |
| | Noticeably more curl | More frizz |
| | definition and regularity | |
| | Noticeably Smoother | Frizzier ends |
| | More curl elongation | Feels dry |
| Dry | More curl definition and | More coated |
| Attributes | regularity | |
| | More frizz control | More dry and frizzy |
| | Noticeably smoother | Lack of sealed ends |
| | Better slip | Noticeably more shine |
| | Better sealed ends | Rougher texture |
| | More noticeable | |
| | transformation | |

### Example 3

### Comparative Composition

Inventive Composition B of Example 1 was compared with a popular commercial benchmark product (Commercial Benchmark #2), known for its excellent conditioning, smoothing, and anti-frizz properties. The Commercial Benchmark #2, like Inventive Composition A, is an oil-based product. However, it lacks cationic surfactants and fatty alcohols. The ingredients for the Commercial Benchmark #2 are shown below.
Refined Sunflower oil, Huile D'Olive Raffinee, Jojoba virgin oil, refined coconut oil, conctrate of alpha-tocopherol (vitamin E), castor oil, nigella oil, polymerized linseed oil, and fragrance.

After shampooing the hair of a volunteer, excess water was squeezed from the hair and the hair was towel dried. While still damp, equal amounts of Inventive Composition A and the Commercial Benchmark #2 were applied to opposite sides of a volunteer's head of hair. Both compositions were spread evenly throughout the hair from roots to tip. An expert evaluator then evaluated the attributes of the treated hair. Without rinsing the composition from the hair, the hair was dried using a blow dryer. After the hair was completely dried, the same expert evaluator again evaluated the attributes of the treated hair. The results are reported in the table below.

| | Inventive A | Benchmark #2 |
|---|---|---|
| Wet | Noticeably easier to | More topical |
| Attributes | distribute | |
| | Noticeably more curl | More frizz |
| | definition and regularity | |
| | Noticeably smoother | Uneven curls |
| | Uniform volume | Feels dry |
| Dry | Smoother | Feels and looks dry |
| Attributes | Uniform shape | More frizzy |
| | Sleeker | Good coating of ends |
| | More sealed ends | Feels hotter during blow |
| | | drying |
| | Softer | |

The foregoing disclosure illustrates and describes embodiments of the invention. The disclosure shows and describes only the preferred embodiments, but it is understood that the invention is useable in various other combinations, modifications, and environments and is capable of changes or modifications within the scope of the inventive concepts as expressed herein, commensurate with the above teachings and/or the skill or knowledge of the relevant art. The embodiments described herein above are further intended to explain best modes known by applicant and to enable others skilled in the art to utilize the disclosure. Accordingly, the description is not intended to limit the invention.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the claimed invention.

The terms “a,” “an,” and “the” are understood to encompass the plural as well as the singular.

Thus, the term “a mixture thereof” or “a combination thereof” also relates to “mixtures thereof” or “combinations thereof.” Throughout the disclosure, the term “a combination thereof” may be used following a list of elements as shown in the following example where letters A-F represent the elements: “one or more elements selected from the group consisting of A, B, C, D, E, F, and a combination thereof.” The term, “a combination thereof” does not require that the combination include all of A, B, C, D, E, and F (although all of A, B, C, D, E, and F may be included). Rather, it indicates that a combination of any two or more of A, B, C, D, E, and F can be included. In other words, it is equivalent to the phrase “one or more elements selected from the group consisting of A, B, C, D, E, F, and a combination of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a combination thereof,” it indicates that that one or more of A, B, C, D, and F may be included, one or more of a salt of A, a salt of B, a salt of C, a salt of D, a salt of E, and a salt of F may be include, or a mixture of any two of A, B, C, D, E, F, a salt of A, a salt of B, a salt of C, a salt of D, a salt of E, and a salt of F may be included.

The term “anhydrous” as used herein refers to a substantial absence of water. An “anhydrous” composition, including the anhydrous hair treatment composition of the instant disclosure comprises less than 5 wt. % water, preferably less than 3 wt. % water, more preferably less than 2 wt. % water. The anhydrous hair treatment composition may comprise less than 1 wt. % water, less than 0.5 wt. % water, or less than 0.1 wt. % of water.

The surfactants referred to throughout the disclosure may include those having a counter-ion such as bromide, chloride, fluoride, hydroxide, nitrate, acetate, benzoate, or sulfate. This list of counterions, however, is non-limiting.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions can be modified in all instances by the term “about,” meaning within +/−10% of the indicated number. For example, an amount of “about 1 wt.” can include an amount as low as 0.90 wt. % or as high as 1.1. Similarly, an amount of “about 50” can include an amount as low as 45 wt. % and as high as 55 wt. %.

Some of the various categories of ingredients identified for the anhydrous hair treatment composition may overlap. In such cases where overlap may exist and the anhydrous hair treatment composition includes two overlapping ingredients (or more than two overlapping ingredients), an overlapping ingredient does not represent more than one claimed component. For example, menthol may be construed as both an “cooling agent” and a “terpene.” If a claimed anhydrous hair treatment composition includes both a cooling agent and a terpene, the menthol in the anhydrous hair treatment composition will serve as only the cooling agent or as only the terpene. Menthol cannot simultaneously serve as both the cooling agent and the terpene.

All percentages, parts, and ratios herein are based upon the total weight of the compositions of the present invention, unless otherwise indicated.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub-ranges between, the given ranges. Thus, a range from 1-5, includes specifically 1, 2, 3, 4 and 5, as well as sub ranges such as 2-5, 3-5, 2-3, 2-4, 1-4, etc.

A “rinse-off” product refers to a composition that is rinsed and/or washed from the hair with water either after or during the application of the composition onto the hair, and before drying and/or styling the hair. At least a portion of the composition is removed from the hair during the rinsing and/or washing.

A “leave-on” product refers to a composition that is not rinsed and/or washed from the hair after or during application of the composition onto the hair. The composition remains on the hair during drying and/or throughout styling.

All components positively set forth in the instant disclosure can be negatively excluded from the claims and from the compositions and methods described throughout the disclosure. In other words, the cleansing compositions of the instant disclosure may be free or essentially free of any one or more of the components positively set forth in the instant disclosure for possible inclusion into the cleansing compositions.

The term “substantially free” or “essentially free” as used herein means the specific material may be present in small amounts that do not materially affect the basic and novel characteristics of the claimed invention. For instance, there may be less than 1% by weight of a specific material added to a composition, based on the total weight of the compositions (provided that an amount of 1% or less by weight does not materially affect the basic and novel characteristics of the claimed invention). Similarly, when a composition is essentially free from a particular element, the composition may include 1 wt. % or less, 0.5 wt. % or less, 0.1 wt. % or less, 0.05 wt. % or less, 0.01 wt. % or less, or none of the specified material. For example, if a composition is “substantially free” or “essentially free” from ethanol, the composition may optionally include ethanol in an amount up to 1 wt. %, based on the total weight of the composition, provided an amount of 1 wt. % does not materially affect the basic and novel characteristics of the composition. Furthermore, the composition may optionally include 0.5 wt. % or less, 0.1 wt. % or less, 0.05 wt. % or less, 0.01 wt. % or less, or none of the ethanol.

All publications and patent applications cited throughout the disclosure are incorporated herein by reference in their entirety, and for any and all purposes, as if each individual publication or patent application were specifically and individually indicated to be incorporated by reference. In the event of an inconsistency between the present disclosure and any publications or patent application incorporated herein by reference, the present disclosure controls.

1. An anhydrous hair treatment composition comprising:
(a) about 10 to about 95 wt. % of one or more vegetable oils, wherein one of the one or more vegetable oils is castor oil, in an amount of at least 10 wt. %;
(b) about 0.1 to about 10 wt. % of one or more cationic surfactants;
(c) about 0.1 to about 10 wt. % of one or more fatty alcohols; and
(d) optionally, about 0.1 to about 5 wt. % of one or more sensorially active agents;
wherein the composition is essentially free from mono-alcohols having from 1 to 6 carbon atoms, polyhydric alcohols having from 1 to 6 carbon atoms, and esters of alpha hydroxy acids; and
all weight percentages are based on a total weight of the composition.

2. The anhydrous hair treatment composition of claim 1, wherein the composition is a homogenous, transparent solution.

3. The anhydrous hair treatment composition of claim 1, wherein the composition forms a lamellar structure when contacted with water.

4. The anhydrous hair treatment composition of claim 1, wherein the composition further comprises, in addition to castor oil, one or more vegetable oils selected from coconut oil, arachis oil, amaranth seed oil, apricot oil, argan oil, avocado oil, babassu oil, cottonseed oil, borage seed oil, camelina oil, camellia oil, safflower oil, peanut oil, pomegranate seed oil, grapefruit seed oil, hemp oil, hazelnut oil, elderberry seed oil, currant seed oil, jojoba oil, linseed oil, macadamia nut oil, corn oil, almond oil, manila oil, evening primrose oil, olive oil, palm oil, palm kernel oil, brazil nut oil, pecan nut oil, peach kernel oil, rapeseed oil, rosehip oil, sea buckthorn fruit oil, sea buckthorn kernel oil, sesame oil, soybean oil, sunflower oil, grapeseed oil, walnut oil, wild rose oil, or wheat germ oil.

5. The anhydrous hair treatment composition of claim 4, wherein one of the one or more vegetable oils is coconut oil.

6. The anhydrous hair treatment composition of claim 1, wherein in (b) and (c) are in a weight ratio of about 1:12 to about 2:1 ((b):(c)).

7. The anhydrous hair treatment composition of claim 1, wherein the one or more cationic surfactants are selected from quaternary ammonium surfactants and amidoamine surfactants.

8. The anhydrous hair treatment composition of claim 1, wherein at least one of the one or more cationic surfactants is selected from amidoamine surfactants.

9. The anhydrous hair treatment composition of claim 8, wherein the at least one amidoamine surfactant is oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethylamine, behenamidoethyldimethylamine, arachidamidopropyldimethylamine, arachidamido-propyidiethylamine, arachidamidoethyidiethylamine, or arachidamidoethyidimethylamine.

10. The anhydrous hair treatment composition of claim 1, wherein the one or more fatty alcohols are selected from fatty alcohols having from 12 to 34 carbon atoms and are branched or linear, and saturated or unsaturated.

11. The anhydrous hair treatment composition of claim 9 comprising a plurality of fatty alcohols, wherein the plurality of fatty alcohols comprises:
one or more solid fatty alcohols selected from stearyl alcohol, cetyl alcohol, cetearyl alcohol alcohol, myristyl alcohol, behenyl alcohol, and arachidyl alcohol; and
one or more liquid fatty alcohols selected from isostearyl alcohol, oleyl alcohol, octyldodecanol, linoleyl alcohol, linolenyl alcohol, lauryl alcohol, 2-hexyldecanol, 2-heptyldecanol, enanthic alcohol, caprylic alcohol, pelargonic alcohol, and capric alcohol.

12. The anhydrous hair treatment composition of claim 1, wherein the composition further comprises one or more emollients selected from fatty ethers, fatty esters, fatty carbonates, propylene glycol esters of fatty acids, and caprylic/capric triglyceride.

13. The anhydrous hair treatment composition of claim 1 comprising the one or more sensorially active agents.

14. The anhydrous hair treatment composition of claim 13, wherein the one or more sensorially active agents are selected from essential oils, cooling agents, terpenes, and terpenoids.

15. The anhydrous hair treatment composition of claim 14, wherein the composition comprises one or more essential oils selected lavender oil, geranium oil, juniper berry oil, bay leaf oil, lemon oil, mandarin orange oil, orange oil, absinthe oil, African bluegrass oil, ajowan oil, anethi oil, angelica root oil, anise star oil, arborvitae wild oil, armoise mugwort oil, asafetida oil, Australian balm oil, basil oil, benzoin oil, bergamot calabrian oil, betel leaf oil, birch sweet oil, birch tar oil, black currant-seed oil, blood orange oil, buchu oil, cabreuva oil, cade oil, cajeput oil, calamus root oil, camphor oil, cananga oil, caraway oil, cardamom oil, carrotseed oil, cassia oil, cedarleaf oil, cedarwood atles oil, cedarwood Chinese oil, cedarwood Himalayan oil, cedarwood texas oil, cedarwood Virginian oil, celery seed oil, chamomile German oil, chamomile moroc oil, chamomile roman oil, chaulmogra oil, chenopodium oil, chilly seed oil, cinnamon bark oil, cinnamon leaf oil, citral oil, citronelia Ceylon oil, clarysage oil, clementine oil, clove bud oil, clove leaf oil, cognac oil, copaiba balsam oil, coriander oil, costus root oil, coffee oil, cumin oil, curcuma oil, curry leaf oil, cypress Australian blue oil, cypress French cypriol oil, davana oil, dill seed oil, dill weed oil, elemi oil, emu oil, eucalyptus oil, fennel bitter oil, fenugreek oil, fir balsam oil, fir needle oil, flax seed organic oil, frankincense oil garlic oil, galbanum oil, ginger oil, ginger grass oil, gaia oil, grapefruit pink oil, grapefruit white oil, guaiacwood oil, helichrysum oil, horseradish oil, hydacheim oil, hyssop oil, jamarosa root oil, Himalayan wild oil, karanj seed oil, kanuka oil, katrafay oil, key lime oil, kunzea oil, kukuinut oil, lantana oil, laurel leaf oil, lemongrass oil, lemon eucalyptus oil, lemon myrtle oil, lemon 5 fold oil, lican oil, lime oil, Litsea cubeba oil, lovage leaf oil, mace oil, mandarin oil, manuka oil, marjoram oil, Melissa leaf oil, menthol liquid oil, mullein oil, myrrh oil, myrtle oil, neem oil, neroli oil, nerolina Australian oil, niaouli oil, noni oil, nutmeg oil, oat oil, onion oil, opoponax oil, orange bitter oil, orange sweet oil, origanum oil, orange 5 fold oil, palmarosa oil, parsley oil, patchouli oil, pennyroyal oil, pepper oil, peppermint oil, peru balsam oil, petitgrain oil, pimento leaf oil, pine long leaf oil, pine scotch oil, pine commercial oil, pomegranate oil, pomouwood oil, ravensara wild oil, rosalina Australian oil, rose geranium oil, rosemary oil, rosewood oil, rue oil, sage Dalmatian oil, sandalwood oil, savine oil, savory winter oil, sea buckthorn oil, spearmint oil, spikenard oil, spruce oil, sugandh kokila oil, sumac oil, tagetes oil, tangerine oil, tansy blue oil, tarragon oil, tangerine 5 fold oil, tea tree oil, tea tree lemon oil, thyme oil, tolu balsam oil, turmeric oil, valerian root oil, vanilla 10 fold oil, vetiver oil, wintergreen oil, wild verbena oil, wormwood oil, yarrow oil, and ylang ylang oil.

16. The anhydrous hair treatment composition of claim 14, wherein the composition comprises one or more cooling agents selected from menthol, menthone glycerol acetal, menthyl lactate, menthyl-3-carboxylic acid N-ethylamide, 2-isopropyl-N-2,3-trimethylbutanamide, 3-menthoxypropane-1,2-diol, 2-hydroxtethyl menthyl carbonate, 2-hydroxypropyl menthyl carbonate, or isopulegol.

17. An anhydrous hair treatment composition comprising:
(a) about 25 to about 75 wt. % of one or more vegetable oils, wherein one of the one or more vegetable oils is castor oil, in an amount of at least 10 wt. %;
(b) about 0.1 to about 5 wt. % of one or more one or more cationic surfactants selected from oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethylamine, behenamidoethyldimethylamine, arachidamidopropyldimethylamine, arachidamido-propyidiethylamine, arachidamidoethyidiethylamine, and arachidamidoethyidimethylamine;
(c) about 1 to about 10 wt. % of a plurality fatty alcohols, wherein the plurality of fatty alcohols comprises:
one or more solid fatty alcohols selected from stearyl alcohol, cetyl alcohol, cetearyl alcohol alcohol, myristyl alcohol, behenyl alcohol, and arachidyl alcohol; and
one or more liquid fatty alcohols selected from isostearyl alcohol, oleyl alcohol, octyldodecanol, linoleyl alcohol, linolenyl alcohol, lauryl alcohol, 2-hexyldecanol, 2-heptyldecanol, enanthic alcohol, caprylic alcohol, pelargonic alcohol, and capric alcohol;
(d) optionally, about 0.1 to about 5 wt. % of one or more sensorially active agents selected from essential oils, cooling agents, terpenes, and terpenoids; and
(e) optionally, about 1 to about 50 wt. % of one or more emollients selected from fatty ethers, fatty esters, fatty carbonates, propylene glycol esters of fatty acids, and caprylic/capric triglyceride; and
(f) optionally, about 0.1 to about 10 wt. % of one or more miscellaneous ingredients;
wherein the composition is essentially free from mono-alcohols having from 1 to 6 carbon atoms, polyhydric alcohols having from 1 to 6 carbon atoms, and esters of alpha hydroxy acids; and
all weight percentages are based on a total weight of the composition.

18. The anhydrous hair treatment composition of claim 17 comprising the one or more emollients of (e), wherein:
the one or more fatty esters are selected from coco-caprylate/caprate, coco-caprylate, coco-caprate, jojoba esters, isopropyl jojobate, ethyl macadamiate, isoamyl laurate, heptyl undecylenate, methylheptyl isostearate, isostearyl isostearate, glyceryl ricinoleate, isostearyl palmitate, myristyl myristate, octyldodecyl myristate, octyldodecyl hydroxystearate, butyl myristate, ethylhexyl cocoate, ethylhexyl palmitate, ethylhexyl stearate, butyl stearate, decyl oleate, isocetyl behenate, isocetyl myristate, isocetyl palmitate, isocetyl stearate, isodecyl oleate, isopropyl isostearate, isopropyl myristate, isopropyl palmitate, oleyl oleate, propylene glycol laurate, octydodecyl erucate, C12-C13 alkyl lactate, C12-C15 alkyl lactate, isostearyl lactate, glycereth-5 lactate, lauryl lactate, myristyl lactate, oleyl lactate, laureth-2 benzoate, C12-C15 alkyl benzoate, C12-C15 pareth-3 benzoate, dipropylene glycol benzoate, isodecyl salicylate, C12-C15 alkyl salicylate, tridecyl salicylate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, laureth-2 ethylhexanoate, cetearyl ethylhexanoate, isodecyl neopentanoate, isostearyl neopentanoate, myristyl neopentanoate, isostearyl behenate, octyldodecyl neopentanoate, and tridecyl neopentanoate;
the one or more fatty ethers are selected from dicaprylyl ether, didecyl ether, dimyristyl ether, distearyl ether, dilauryl ether, dihexyl ether, dioctyl ether, diethylhexyl ether, dioctyl ether, and dodecyl ether; and
the one or more fatty carbonates are selected from C14-15 dialkyl carbonate, dicaprylyl carbonate, diethyl carbonate, dihexyl carbonate, diethylhexyl carbonate, dimethoxyphenyl phenyloxoethyl ethylcarbonate, dimethyl carbonate, dipropyl carbonate, dipropylheptyl carbonate, and dioctyl carbonate.

19. The anhydrous hair treatment composition of claim 18 comprising a plurality of emollients, wherein the plurality of emollients comprises:
(i) about 5 to about 15 wt. % of caprylic/capric triglyceride;
(ii) about 1 to about 10 wt. % of one or more fatty esters;
(iii) about 2 to about 15 wt. % of one or more fatty ethers; and
(iv) about 1 to about 10 wt. % of one or more fatty carbonates.

20. A method for treating hair comprising applying the anhydrous hair treatment composition of claim 1 to the hair.
